(19) United States
(12) Patent Application Publication
(10) Pub. No.: US 2018/0127399 A1
(43) Pub. Date: May 10, 2018

(54) BIOSOURCED COMPOUND HAVING EPOXIDE FUNCTIONS, METHOD FOR THE SYNTHESIS OF SUCH A COMPOUND, AND USE THEREOF FOR PRODUCING EPOXY RESIN

(71) Applicants: INSTITUT NATIONAL DE LA RECHERCHE AGRONOMIQUE, PARIS CEDEX 07 (FR); INSTITUT NATIONAL D'ETUDES SUPERIEURES AGRONOMIQUES DE MONTPELLIER (MONTPELLIER SUP AGRO), MONTPELLIER (FR)

(72) Inventors: LAURENT ROUMEAS, MONTPELLIER (FR); HÉLÈNE FULCRAND, SAINT GELY DU FESC (FR); CHAHINEZ AOUF, MONTPELLIER (FR); ERIC DUBREUCQ, MONTPELLIER (FR)

(21) Appl. No.: 15/570,325

(22) PCT Filed: Apr. 22, 2016
(86) PCT No.: PCT/FR2016/050957

§ 371 Date: Oct. 27, 2017

(30) Foreign Application Priority Data
Apr. 27, 2015 (FR) 1553769

(51) Int. Cl. C07D 407/04; C07D 407/14; C07D 207/30; C07D 241/40; C07D 203/04; C08G 59/06; C08G 59/24; C08G 59/32; C08G 59/50

(52) U.S. Cl. CPC C07D 407/04; C07D 407/14; C07D 207/30; C07D 241/40; C08G 59/504; C08G 59/06; C08G 59/24; C08G 59/3281; C07D 203/04

(57) ABSTRACT

A compound has at least one epoxide function which can be synthesized by an epoxidation reaction of a compound having at least one hydroxyl function obtained by depolymerization of condensed tannins by a nucleophile selected from furan and the derivatives thereof. The compound having at least one epoxide function has properties that are useful in the production of epoxy resins, as a synthon precursor and/or as a starting material for forming a polyamine hardener.

The present invention falls within the field of the production of epoxy resins. More particularly, the invention relates to a compound having one or more epoxide function(s), that can be obtained from renewable resources, and also to a method for synthesizing such a compound. The invention also relates to the use of such a compound having one or more epoxide function(s) for producing an epoxy resin, and also to a method for producing an epoxy resin from such a compound having one or more epoxide function(s), to the epoxy resin thus obtained and to the use thereof. The invention also relates to a curing agent having one or more amine function(s), that can be obtained from a compound having one or more epoxide function(s) according to the invention, and that can be used for producing said epoxy resin.

Epoxy resins represent an important class of polymers, in particular polymers of the thermosetting type. Epoxy resins are currently used in a wide range of applications, in particular as materials, coatings, paints, varnishes, adhesives, glues, etc., in many sectors such as the electronics sector (in particular for insulating components), the materials sector and the aerospace sector. These resins have indeed excellent mechanical and electrical properties, and also excellent properties of resistance both to friction and to temperature variations and to numerous chemical products.

Epoxy resins are conventionally synthesized by mixing two components, one of which is a component commonly known as epoxide prepolymer, which constitutes the precursor synthon for the resin, and one of which is a curing agent, optionally in the presence of additives, in particular of a reaction accelerator or catalyst. Schematically, the curing agent has the function of reacting with the epoxide functions of the prepolymer so as to ensure crosslinking of the resin, by formation of a three-dimensional network.

Industrially, the synthesis of aromatic epoxide prepolymers is essentially carried out based on a petrochemistry-derived product, bisphenol A. However, the increased scarcity of oil resources and the considerations relating to the quality of the environment and to human health, associated in particular with the pollution caused by the chemical industry, push researchers and those involved in this industry to turn to the use of materials that originate from renewable resources, in particular derived from biomass, and that are environmentally friendly, in particular of biobased phenolic compounds capable of replacing the phenolic petrochemical compounds such as bisphenol A.

Condensed tannins, also known as proanthocyanidins, constitute a source that is particularly interesting, since it is abundant, of such phenolic compounds capable of constituting starting products for forming epoxide prepolymers. Indeed, condensed tannins are phenolic biopolymers that are essentially present in rapidly growing and rapidly renewed soft tissues, such as leaves, stems, etc. This class of polyphenols is the most abundant after that of lignins. Contrary to lignins, which are constituents of lignocelluloses, these being structuring elements of secondary walls of plant cells, condensed tannins are stored in the cell vacuoles in the form of organelles, and are thus readily extractable. These compounds are found in particular in many available and varied natural resources, such as food-processing residues, for example in fruit marcs, and non-exploited biomass, in particular in the bark, the leaves and the needles of trees, vines, fruits, etc.

It has thus been proposed by the prior art to replace, for the formation of epoxy resins, bisphenol A with phenolic compounds based on renewable carbon found in vegetable biomass. By way of example of such prior art, document FR-A-2 946 049 describes, inter alia, a method for producing an epoxy-type resin from epoxidized phenolic compounds (epoxide prepolymers), which are themselves obtained by epoxidation of compounds resulting from the depolymerization of condensed tannins by a nucleophilic reagent chosen from compounds having a thiol function and monoaromatic phenolic compounds. However, the synthesis of epoxide prepolymers from condensed tannin depolymerization products, as proposed in said document, does not give satisfactory results. The depolymerization products formed by the extension units prove in fact to be too unstable in an alkaline medium to make it possible to actually perform the synthesis of the epoxide prepolymer.

The present invention aims to provide a compound having one or more epoxide function(s) which can actually be obtained from renewable resources, in particular from biomass, and more particularly from condensed tannins, and which can be used, as a replacement for phenolic petrochemical compounds such as bisphenol A, for the production of epoxy resins, the latter having moreover properties comparable to those of the resins obtained based on such petrochemical compounds.

An additional objective of the invention is to provide a method for obtaining such a compound from biobased substances, and a method for producing an epoxy resin from such a compound, these methods being environmentally friendly, simple, fast and inexpensive to carry out.

Thus, according to a first aspect, the present invention relates to a compound having one or more epoxide function(s), of the type glycidyl ether having one or more terminal epoxide function(s), and derived from flavonoids, in which a flavonoid residue is bonded by covalent bonding, at the level of the pyran ring, to a furan derivative. This compound corresponds to general formula (I):

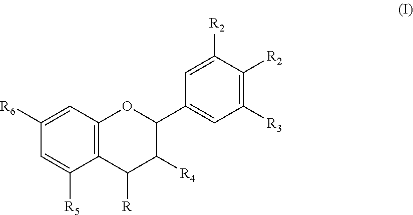

wherein:
R₁, R₂ and R₃, which may be identical or different, each represent a hydrogen atom, a group of formula (III), or a group —OR₉ where R₉ represents a protective group for a hydroxyl function, said group of formula (III) corresponding to the formula:

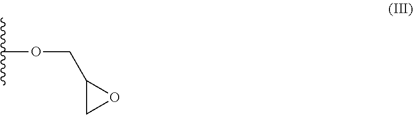 [SMILES: *OCC1CO1]

and/or (R₁ and R₂) together represent a group of general formula (IV) or (R₂ and R₃) together represent a group of general formula (IV):

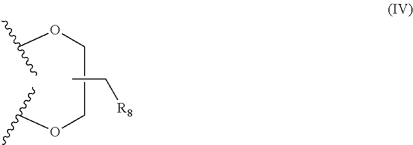

wherein R₈ represents a hydroxyl group, a group of formula (III), or a group —OR_{9′} where R_{9′} represents a protective group for a hydroxyl function,
R₄ represents a hydrogen atom, a hydroxyl group, optionally protected by a protective group for a hydroxyl function, a group of formula (III) or a group —OR₇, wherein R₇ represents a group of general formula (II):

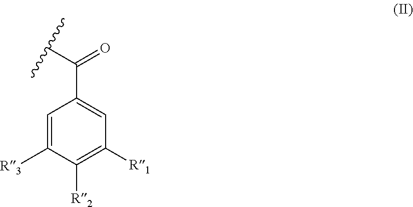

wherein R″₁, R″₂ and R″₃, which may be identical or different, each represent a group of formula (III), or a group —OR_{9″}, where R_{9″} represents a protective group for a hydroxyl function,
R₅ represents a hydrogen atom, a group of formula (III), or a group —OR_{9″}, where R_{9′″} represents a protective group for a hydroxyl function,
R₆ represents a group of formula (III), or a group —OR_{9′″} where R_{9′″} represents a protective group for a hydroxyl function, and
R represents a furan ring, which may optionally be substituted,
at least one substituent among R₁, R₂, R₃, R₄, R₅, R₆, R₈, R″₁, R″₂ and R″₃ representing a group of formula (III).

The invention also relates to any salt of said compound of general formula (I).

The term “protective group for a hydroxyl function” is intended to mean any group conventionally used in itself to protect a hydroxyl function, in particular a phenolic hydroxyl, that is to say to mask its reactivity with a view to subsequent reactions. Each of the protective groups for a hydroxyl function can for example be chosen from alkyl, acyl, in particular acetyl, benzyl, silyl, sulfonyl, etc., groups. The protective groups for a hydroxyl function that are borne by the compound according to the invention may all be identical, or be different than one another, the protective groups borne by the hydroxyl functions of one and the same nucleus then preferably being identical to one another.

The group R, derived from a furan nucleus, of general formula (VII′):

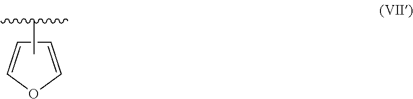

can equally be substituted or unsubstituted.

The bond to the pyran ring can be borne by any one of its carbon atoms.

The furan nucleus can be substituted, on one, two or three of its carbon atoms not bearing the covalent bond with the pyran ring. Each of its carbon atoms can bear any type of substituent, depending on the properties that it is desired to confer on the compound of formula (I), and more particularly on the epoxy resin for the production of which it is intended to be used. Preferably, none of the substituents borne by the furan nucleus comprises a function capable of reacting with an epoxide function.

In particular, the group R can correspond to general formula (VII″):

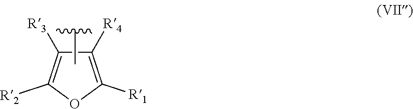

wherein, with the limit that one substituent among R′₁, R′₂, R′₃ and R′₄ represents the covalent bond with the pyran ring, R′₁, R′₂, R′₃ and R′₄, which may be identical or different, each represent:
hydrogen atom,
a linear, branched and/or cyclic carbon-based radical which can comprise a single ring or several condensed rings, which may be saturated and/or unsaturated, optionally aromatic, and optionally substituted, and which optionally comprises one or more heteroatoms and/or one or more groups comprising one or more heteroatoms, each heteroatom being in particular chosen from O, N, P, Si and S,
a carbonyl, thiocarbonyl or amide group,
a halogen atom, such as a fluorine, chlorine, bromine or iodine atom,
a nitro group, or
a sulfonate or sulfonamide group.

Preferably, none of the substituents among R′₁, R′₂, R′₃ and R′₄ contains any amine, anhydride, carboxylic acid, phenol, thiol or sulfonic acid function.

Two substituents among R′₁, R′₂, R′₃ and R′₄ may together form an additional ring, fused to the furan nucleus.

The compound according to the invention can correspond to general formula (I′):

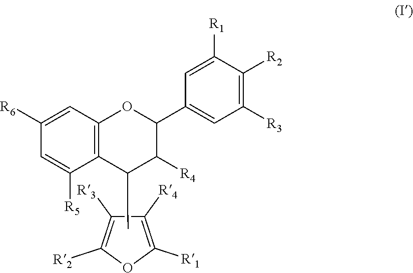

wherein R₁, R₂, R₃, R₄, R₅, R₆, R′₁, R′₂, R′₃ and R′₄ are as defined above, or can be a salt thereof.

The compound having one or more epoxide function(s) according to the invention has many applications, and advantageously constitutes a novel family of platform molecules that can enable the synthesis of numerous polymers such as, for example, polyepoxides, polyesters, polycarbonates, polyurethanes, vinyl esters, polyamides, etc.

This compound can in particular be used for the production of resins of epoxy type. It makes it possible to obtain epoxy resins which have particularly advantageous mechanical properties, in particular properties similar to those of Plexiglas® in terms of mechanical strength.

Entirely advantageously, its degree of glycidylation can advantageously be controlled, thus making it possible to control its reactivity and the structure of the epoxy resin that it makes it possible to obtain.

General formula (I) encompasses all the possible combinations of isomer forms at asymmetric carbons, and all the mixtures of such isomer forms. Each particular isomer can be obtained from a mixture of isomers by purification methods that are in themselves conventional for those skilled in the art.

The compound according to the invention can in particular be such that, in general formula (VII″), or else in general formula (I′), at least one substituent among R′₁, R′₂, R′₃ and R′₄ represents a hydrogen atom.

Preferentially, R′₁, R′₂, R′₃ and R′₄, which may be identical or different, each represent, when they do not form the covalent bond with the flavonoid residue, a hydrogen atom or a linear or branched, optionally substituted hydrocarbon-based radical optionally interrupted with one or more heteroatoms and/or with one or more groups comprising one or more heteroatoms, each heteroatom being possibly for example chosen from O, N, P, Si and S, said hydrocarbon-based radical preferably not comprising any amine, anhydride, carboxylic acid, phenol, thiol or sulfonic acid function.

Compounds according to the invention can in particular correspond to general formulae (Ia) and (Ib) below:

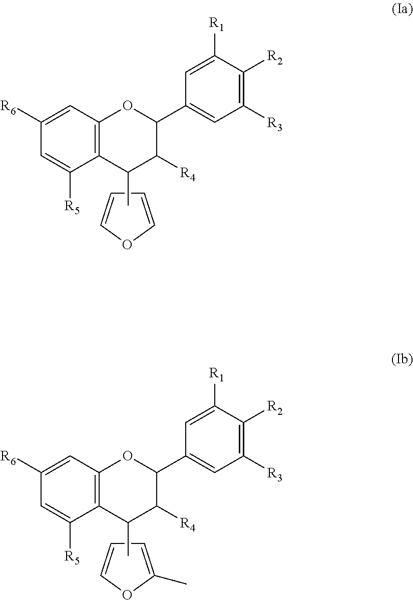

in which formulae, R₁, R₂, R₃, R₄, R₅ and R₆ are as described above with reference to general formula (I).

In variants of the invention, in general formula (VII″), or else in general formula (I′), R′₁, R′₂, R′₃ and R′₄ correspond in particular to one or more of the characteristics below:
R′₁ represents the covalent bond with the pyran ring, and R′₃ and R′₄ each represent a hydrogen atom,
R′₂ represents a hydrogen atom, or R′₂ represents a methyl radical.

In particular, in general formula (VII″), or else in general formula (I′), all three of R′₂, R′₃ and R′₄ can represent a hydrogen atom, and R′₁ can represent the covalent bond with the pyran ring. The compound according to the invention then corresponds to general formula (Ic):

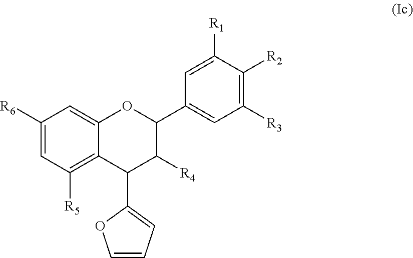

wherein R₁, R₂, R₃, R₄, R₅ and R₆ are as defined above with reference to general formula (I).

The expression “furylated compound having one or more epoxide function(s)” will be used in the present description to designate such a compound.

In variants of the invention, R₁, R₂, R₃, R₄, R₅ and R₆, and R′₁, R′₂, R′₃ and R′₄, are as defined above, and at least one group among R′₁, R′₂, R′₃ and R′₄ represents neither a hydrogen atom, nor the covalent bond with the pyran ring.

The compound having one or more epoxide function(s) according to the invention can in particular be such that R′₃ and R′₄ each represent a hydrogen atom, R′₁ represents the covalent bond with the pyran ring, and R′₂ represents a methyl radical. The compound then corresponds to general formula (Id) below, wherein R₁, R₂, R₃, R₄, R₅ and R₆ are as defined above:

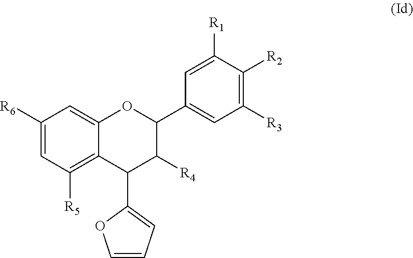

The expression “sylvanylated compound having one or more epoxide function(s)” will be used in the present description to designate such a compound.

According to a particular characteristic of the invention, in general formula (I), at least two substituents, preferably at least three substituents, and preferentially at least four substituents, among R₁, R₂, R₃, R₄, R₅, R₆, R₈, R″₁, R″₂ and R″₃, represent a group of formula (III). By way of example, two, or three, or four, or five, or six, or seven substituents, among R₁, R₂, R₃, R₄, R₅, R₆, R₈, R″₁, R″₂ and R″₃, represent a group of formula (III).

Examples of compounds having one or more epoxide function(s) in accordance with the invention correspond to general formulae (Ie) to (Ig) below:

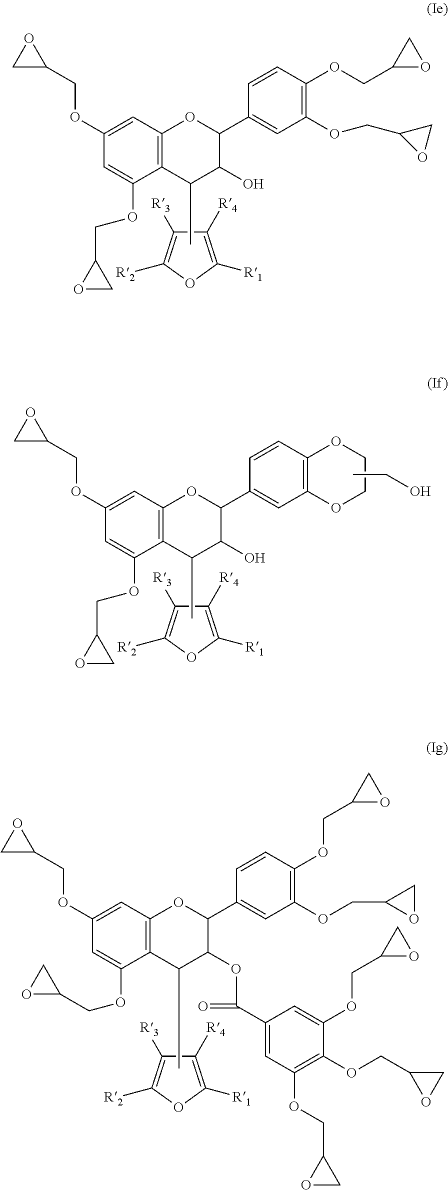

wherein R′₁, R′₂, R′₃ and R′₄ are as described above.

Another aspect of the invention relates to a method for synthesizing a compound having one or more epoxide function(s) according to the invention, corresponding to one or more of the characteristics above. This method comprises a step of epoxidation of a compound of general formula (V):

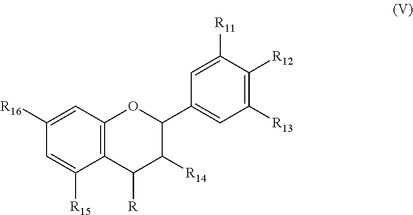

wherein:
R₁₁, R₁₂, R₁₃ and R₁₅, which may be identical or different, each represent a hydrogen atom or a hydroxyl group, optionally protected with a protective group for a hydroxyl function,
R₁₄ represents a hydrogen atom or a group —OR₁₇, wherein R₁₇ represents a hydrogen atom, a protective group for a hydroxyl function or a group of general formula (II′):

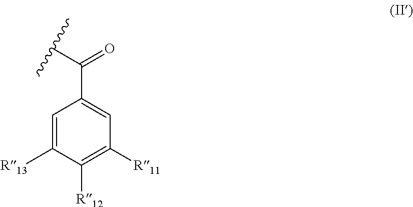

wherein R″₁₁, R″₁₂ and R″₁₃, which may be identical or different, each represent a hydroxyl group, optionally protected with a protective group for a hydroxyl function,
R₁₆ represents a hydroxyl group, optionally protected with a protective group for a hydroxyl function, and
R represents a furan nucleus, which may optionally be substituted,
at least one substituent among R₁₁, R₁₂, R₁₃, R₁₄, R₁₅, R₁₆, R″₁₁, R″₁₂ and R″₁₃ representing a free hydroxyl group, or
a salt thereof.

The group R can correspond to one or more of the characteristics described above with reference to the compound having one or more epoxide function(s) of formula (I) according to the invention.

This epoxidation step can be carried out by any method that is in itself conventional for those skilled in the art. The reaction conditions thereof are in particular established according to the number of free hydroxyl functions in the compound of general formula (V), which determines the final degree of epoxidation of the compound having one or more epoxide function(s) according to the invention. Such an establishment is within the competence of those skilled in the art. The final degree of epoxidation sets in particular the reactivity of the formed compound having one or more epoxide function(s), and consequently the final mechanical properties of an epoxy resin prepared based on the compound having one or more epoxide function(s) according to the invention, after curing of said epoxy resin.

By way of example, the epoxidation step can be carried out by applying the operating conditions described in the prior art document EP 0 095 609.

More generally, the epoxidation step can be carried out by means of any compound having an epoxide function and having a nucleofuge group, for example by placing the compound of general formula (V) in the presence of glycidol mesylate.

In particular embodiments of the invention, the epoxidation of the compound of general formula (V) is carried out by placing this compound of general formula (V) in the presence of an epihalohydrin, preferably of epichlorohydrin.

Epichlorohydrin offers in particular the advantage of a biobased origin. This compound can indeed be obtained by chlorination, for example by means of the process known as Epicerol®, of glycerol, which is a co-product of the transesterification of vegetable oils for the production of biodiesel, as well as for the production of soap by hydrolytic saponification.

Such a step of epoxidation/glycidylation of the compound of general formula (V) by condensation of epichlorohydrin can be carried out under conventional reaction conditions, in particular in a solvent such as ethanol, and in the presence of a strong base such as sodium hydroxide, or using a phase-transfer catalyst such as benzyltriethylammonium chloride, the epichlorohydrin in this case acting as solvent.

In particular embodiments of the invention, the compound of general formula (V) is obtained by depolymerization reaction of polyfunctional polyaromatic compounds derived from renewable resources, more particularly of condensed tannins, in the presence of an acid and by means of a nucleophile of general formula (VII):

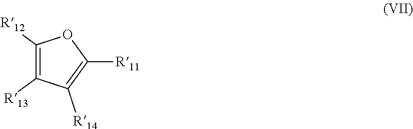

wherein R′₁₁, R′₁₂, R′₁₃ and R′₁₄, which may be identical or different, each represent a hydrogen atom or a substituent not comprising a mesomeric-effect electron-withdrawing group conjugated to the furan nucleus.

In this general formula (VII), at least one substituent among R′₁₁, R′₁₂, R′₁₃ and R′₁₄ represents a hydrogen atom, so as to allow the formation of the covalent bond with the pyran ring of the flavonoid residue of an extension unit of the condensed tannins, leading to the obtaining of a compound of general formula (V) above.

The expression “substituent not comprising a mesomeric-effect electron-withdrawing group conjugated to the furan nucleus” is intended to mean any substituent comprising no electron-withdrawing group which is bonded directly, or by conjugation, to the furan ring of the nucleophile of general formula (VII). It is within the competence of those skilled in the art to determine, on the basis of their general knowledge, which substituents do or do not fall into such a definition. The general knowledge of those skilled in the art on the subject is in particular illustrated by the handbook by René Milcent, Chimie organique: Stéréochimie, entités réactives et reactions [Organic chemistry: Stereochemistry, reactive entities and reactions], EDP Sciences—2007, in particular in chapters 5.5 and 5.6.

By way of example, substituents excluded from the definition of R′₁₁, R′₁₂, R′₁₃ and R′₁₄ are substituents comprising, bonded directly or by conjugation to the furan ring, an electron-withdrawing radical such as a nitro radical, a carbonyl radical, a carboxylic radical or a sulfonic radical, optionally salified or esterified, an amide radical, a cyano radical, a sulfonyl radical, etc.

The nucleophile can in particular be such that, in general formula (VII), R′₁₁, R′₁₂, R′₁₃ and R′₁₄, which may be identical or different, each represent, with the exception of the substituent representing the hydrogen atom allowing the formation of a covalent bond with the pyran ring of the flavonoid residue of an extension unit of the condensed tannins:
a hydrogen atom,
a group comprising an inductive-effect or mesomeric-effect electron-donating radical, bonded directly or by conjugation to the furan ring, for example chosen from an amino radical, an oxy radical or a thio radical, which is optional substituted, said group not comprising any mesomeric-effect electron-withdrawing group conjugated to the furan nucleus, or
a linear, branched and/or cyclic carbon-based radical which can comprise a single ring or several condensed rings, which is saturated and/or unsaturated, optionally aromatic, and optionally substituted, and which optionally comprises one or more heteroatoms and/or one or more groups comprising one or more heteroatoms, each heteroatom being in particular chosen from O, N, P, Si and S.

The condensed tannins are oligomers or polymers of polyfunctional polyaromatic monomers. These monomers belong to the class of flavan-3-ols, of general formula:

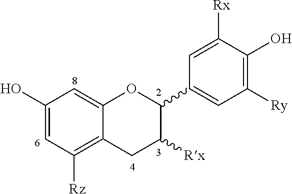

wherein Rx, Ry and Rz, which may be identical or different, each represent a hydrogen atom or a hydroxyl group, and R′x represents a hydrogen atom, a hydroxyl group or a gallate group of formula:

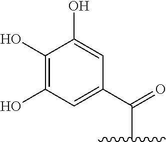 [SMILES: *C(=O)c1cc(O)c(O)c(O)c1]

Depolymerization of the condensed tannins gives in particular the (+)catechin and (−)epicatechin monomers, and also derivatives substituted mainly at C4 and potentially at C2. The C2, C3 and C4 carbon atoms of these derivatives are asymmetrical, and stereoisomers other than those initially present in the structures of tannins can also be formed during the depolymerization reaction.

In order to meet one of the objectives set by the present invention, namely that the method for synthesizing the compound having one more epoxide function(s) according to the invention is as environmentally friendly as possible, and allows the exploitation of local agro-resources, the condensed tannins are preferentially derived from renewable resources such as by-products and coproducts of the agricultural, forestry or wine industry, for example fruit marcs, wood barks, etc., and unexploited biomasses, such as pine needles, dead leaves, etc.

The depolymerization reaction of the method according to the invention can be carried out using condensed tannins which have been previously isolated from biomass. The method then comprises a prior step of extraction of the condensed tannins from biomass, for example from grape seeds. Such an extraction can be carried out by any technique known to those skilled in the art, in particular the techniques illustrated by the publications by Prieur et al., 1994 (Phytochemistry 36, 781-784), and Rigaud et al., 1993 (J. Chromatogr. A 654, 255-260).

Alternatively, the depolymerization reaction can be carried out directly from biomass, without prior extraction of the condensed tannins contained in this biomass, for example directly on a bark fraction, such as a fraction of Pseudotsuga menziesil (Douglas pine) bark.

In preferred embodiments of the invention, the furan derivative of general formula (III), acting as a nucleophile for the condensed-tannin depolymerization reaction, is also biobased. In particular, it may be furan, of general formula (VIIa), or 2-methylfuran, otherwise known as sylvan, of general formula (VIIb), both offering the advantage of a biobased origin:

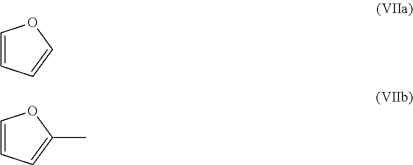

The acid used for the condensed-tannin depolymerization reaction may be of any type. It is in particular chosen from acids commonly used in the industrial field, such as sulfuric acid (H₂SO₄), hydrochloric acid (HCl), methanesulfonic acid (MsOH), formic acid and acetic acid, or a mixture of such acids. Its concentration is preferably equivalent to the concentration of this acid required to confer on an aqueous solution a pH of between −1 and 3.5.

In particular embodiments of the invention, the depolymerization reaction is carried out in a polar solvent, preferably a protic solvent, such as for example methanol, ethanol, formic acid or acetic acid, or in a mixture of solvents containing at least one polar, preferably protic, solvent.

The proportion of the nucleophile of general formula (VII) in the solvent can then be between 1% and 75% by volume, preferably between 10% and 75% by volume, relative to the volume of solvent, and can preferentially be approximately equal to 25% by volume relative to the volume of solvent.

The condensed-tannin depolymerization reaction is preferably carried out at a temperature below or equal to the boiling point of the nucleophile of general formula (VII) at the pressure applied in the reactor, and optionally, when a solvent is added to the reaction medium, at a temperature below or equal to the boiling point of this solvent at this same pressure.

At the end of the condensed-tannin depolymerization reaction, the compound of general formula (V) can be separated from the reaction medium, before carrying out the epoxidation step making it possible to obtain a compound having one or more epoxide function(s) according to the invention. This separation can be carried out by any technique that is conventional in itself. For example, it can consist in adding water to the medium, in evaporating off the solvents and the nucleophile by evaporation under vacuum, and then in extracting the product(s) of interest by liquid/liquid extraction using a water-immiscible organic solvent, such as ethyl acetate, dichloromethane, diethyl ether, etc.

In particularly advantageous variants of the invention, in terms of time and ease of implementation, the epoxidation step is carried out on the reaction crude obtained by the reaction of condensed-tannin depolymerization by means of the nucleophile of general formula (VII), without prior purification of this reaction crude.

In this way, at the end of the epoxidation step, a mixture of compounds having one or more epoxide function(s) according to the invention, comprising in particular derivatives of (+)catechin and of (−)epicatechin, is obtained. The present invention thus also relates to a mixture of compounds comprising one or more epoxide function(s) according to the invention, each corresponding to general formula (I) above, and different than one another by virtue of the position of the substituent(s) bearing an epoxide function.

This mixture of compounds having one or more epoxide function(s) according to the invention can be subjected to a step of separation of the compounds comprising one or more epoxide function(s) obtained from one another, prior to their subsequent use, in particular with a view to the production of an epoxy resin.

Alternatively, this mixture of compounds can be employed directly for such a subsequent use.

The method according to the invention for synthesizing a compound having one or more epoxide function(s), or a mixture of such compounds, is particularly simple to carry out, this being at low cost. It also makes it possible to obtain this or these compound(s) with a high yield, and from renewable natural resources.

By way of example, such a synthesis method can be carried out as follows, according to the operating conditions described in the prior document EP 0 095 609. The compound of general formula (V) above, or the products resulting from the depolymerization of tannins by a nucleophile of general formula (VII) above, for example by furan or sylvan, are dissolved in the glycidylation agent, in particular an epihalohydrin, for example epichlorohydrin, in excess. A catalyst, for example benzyltriethylammonium chloride, is added in catalytic amount. The reaction is left to continue for approximately 1 h at approximately 100° C. A concentrated aqueous sodium hydroxide solution is then added, as is additional benzyltriethylammonium chloride. The reaction is left to continue for a further approximately 1 h 30, at approximately 30° C.

The reaction product can then be subjected to various steps of purification, in a manner conventional in itself, so as to obtain the compound having one or more epoxide function(s) of general formula (I) according to the invention, or a mixture of such compounds. This or these compound(s) is (are) typically in the form of an oil.

Prior to the step of epoxidation of the compound of general formula (V), above, the synthesis method according to the invention can comprise a step of partial protection of possibly free hydroxyl functions of this compound, in particular functions borne by aromatic nuclei, so as to control the subsequent degree of glycidylation of the obtained compound having one or more epoxide function(s).

By way of example, when the compound of general formula (V) is catechin, the hydroxyl functions of the catechol nucleus can thereby be selectively protected according to the following strategies of protection with the protective groups listed below, which then prove to be specific for the catechol nucleus. These strategies have been successfully applied by the present inventors prior to the implementation of a synthesis method according to the invention, for protecting the hydroxyl functions of the catechol nucleus by:
formation of a cyclic ketal, by ketalization or trans-ketalization, more specifically in the form of a acetonide, as described in document US 2013/0030192;
formation of 2-BOC-ethylidene (“bocdene”) or 2-Moc-ethylidene (“mocdene”) derivatives, as described in the publication by Ariza et al. 2001 (Organic Letters 3, 1399-1401);
formation of a dioxolane (oxole) by methylenation, as described in the publication by Clark et al., 1976 (Tetrahedron Letters 17, 3361-3364);
formation of a cyclic borate, as described in the publication by Scheline, 1966 (Acta Chem. Stand. 20, 1182);
formation of a cyclic ortho-formate, more specifically in the form of ethyl ortho-formate, as described in the publication by Merz et al., 1993 (Synthesis 8, 797-802).

Another aspect of the invention lies in the use of a compound having one or more epoxide function(s) according to the invention, corresponding to one or more of the characteristics above, or where appropriate of a mixture of such compounds, for the production of an epoxy resin.

As previously set out, the synthesis of epoxy resins conventionally uses two components, which are mixed with one another to form the crosslinked resin, namely an epoxide prepolymer, which constitutes the precursor synthon for the resin, and a curing agent.

The compound having one or more epoxide function(s) according to the invention, in the embodiments in which it comprises at least two epoxide functions, can constitute a precursor synthon for said epoxy resin, that is to say can act as an epoxide prepolymer in the method for producing the epoxy resin.

Beforehand, it may have been subjected to a step, which is optionally selective, of opening of some of its epoxide functions, so as to decrease its subsequent reactivity, and to control the structure of the epoxy resin formed. The opening can be carried out by addition of a nucleophile in an acidic, neutral or basic medium, in an organic or aqueous medium, by chemical or enzymatic catalysis.

Thus, the compound having one or more epoxide function(s) according to the invention, in the embodiments in which it comprises at least two, preferably at least three, epoxide functions, can be subjected, so as to form a compound intended to constitute a precursor synthon for the epoxy resin, to a step of opening of at least one of its epoxide rings by reaction with a nucleophilic reagent, termed nucleophilic modulation reagent, which has a function capable of reacting with said epoxide ring so as to cause opening thereof, with the exclusion of an amine function, and which has no other group that is reactive with respect to the epoxide functions.

This step, termed step of modulation of the degree of crosslinking, is carried out in such a way as to leave intact at least one, preferably at least two, epoxide rings of said compound having one or more epoxide function(s).

Such a step advantageously makes it possible to modify the number of epoxide functions of the compound having epoxide functions according to the invention, and consequently to modulate the degree of crosslinking of the epoxy resin that will subsequently be formed from this compound.

The expression “nucleophilic reagent having no other group that is reactive with respect to the epoxide functions” is intended to mean that the nucleophilic reagent does not comprise, other than the function capable of reacting with an epoxide ring to cause opening thereof, any other group capable of reacting with an epoxide function in the absence of specific reaction conditions, for example in the absence of specific catalysts, of particular temperature conditions, etc. It is within the competence of those skilled in the art to determine which nucleophilic reagents can be used for the step of modulation of the degree of crosslinking.

As group that is not reactive with respect to an epoxide ring, the nucleophilic modulation reagent according to the invention can comprise any group chosen from aliphatic, aromatic, non-phenolic hydroxyl, ester, amide, nitrile, ether, thioether, sulfone, sulfoxide, halogen, etc., groups. In particular excluded from the invention are nucleophilic reagents comprising, other than the function capable of reacting with an epoxide ring to cause opening thereof, at least one group chosen from phenolic hydroxyls, thiols, acid anhydrides, amines, and acid groups, such as carboxylic acid, sulfonic acid, etc., groups, or basic groups, such as alkoxides, etc.

In particular embodiments of the invention, the compound having one or more epoxide function(s) according to the invention can otherwise, or equally, be subjected to a step of opening of at least one of its epoxide rings, preferably of each of its epoxide rings, this step being carried out so as to ensure at the same time the introduction of an amine function into the molecule, so as to form a curing agent intended to be used for the production of said epoxy resin.

Beforehand, or simultaneously, the compound having one or more epoxide function(s) according to the invention, in the embodiments in which it comprises at least two epoxide functions, can be subjected to a step of modulation of the degree of crosslinking as described above, that is to say of opening of at least one of its epoxide rings by reaction with a nucleophilic modulation reagent having a function capable of reacting with said epoxide ring to cause opening thereof, with the exclusion of an amine function, and having no other group that is reactive with respect to the epoxide functions, this step being carried out so as to leave intact at least one epoxide ring of said compound having one or more epoxide function(s) for the reaction of opening of the ring with introduction of an amine function.

Although, as set out above, the methods proposed by the prior art for the synthesis of epoxy resins use two components, namely a precursor synthon and a curing agent, it has been discovered by the present inventors that, entirely surprisingly, the compounds having epoxide functions according to the invention of general formula (I) also make it possible to form such resins in the presence not only of a conventional curing agent, that is to say a crosslinking agent, but also in the sole presence of a simple initiator of a reaction for anionic polymerization of the compound with itself, such as a primary amine. For ease of language, these initiators will be encompassed in the present description under the term curing agent. The compounds having epoxide functions of general formula (I) according to the invention thus make it possible to form epoxy resins in the presence equally of primary and secondary or tertiary amines, for example in the presence of one of the following amines: octylamine, ethanolamine, diethylamine, piperidine, pyrrolidine, pyridine, triethylamine, etc.

The epoxide resins thus obtained can also have, depending on the compounds used for the preparation thereof, entirely advantageously, shape-memory properties, that is to say that:
their glass transition temperature (Tg) is lower than their decomposition temperature,
above their Tg, they exhibit a rubbery (deformable and elastic) behavior,
in this rubbery form, they can be deformed,
if the deformation imposed is maintained during their cooling to a temperature below their Tg, they harden and retain their deformation in a long-lasting manner. During subsequent reheating to a temperature above the Tg, they return to their initial shape,
the hot-deformation/cooling/reheating cycles with return to the initial shape can be repeated without loss of property within the limit of the tearing of the material during the deformation.

These properties prove to be particularly advantageous for certain applications.

The epoxy resins obtained according to the invention can, depending on the compounds used for the preparation thereof, have thermoplastic properties.

An additional subject of the invention is a method for producing an epoxy resin, by mixing at least one epoxide prepolymer and a curing agent, in particular a compound having an amine function. As indicated above, the term “curing agent” encompasses, in addition to the actual crosslinking agents, the initiators of reactions of anionic polymerization of the compound with itself.

According to particular features of the present invention:
the epoxide prepolymer can be a compound having one or more epoxide function(s) in accordance with the invention, comprising at least two epoxide functions, and corresponding to one or more of the characteristics described above, or optionally a mixture of such compounds having epoxide functions;
the epoxide prepolymer can be obtained from a compound having one or more epoxide function(s) in accordance with the invention, comprising at least two, preferably at least three, epoxide functions, by means of a step of opening at least one of its epoxide rings by reaction with a nucleophilic reagent, called nucleophilic modulation reagent, as described above, that is to say having a function capable of reacting with said epoxide ring so as to cause opening thereof, with the exclusion of an amine function, and having no other group that is reactive with respect to the epoxide functions, this step of modulation of the degree of crosslinking being carried out so as to leave intact at least one, preferably at least two, epoxide rings of said compound having one or more epoxide function(s); and/or
the curing agent can be obtained from a compound having one or more epoxide function(s) in accordance with the invention, and corresponding to one or more of the characteristics described above, or optionally from a mixture of such compounds comprising one or more epoxide function(s), by means of a step of opening at least one epoxide ring, preferably each of the epoxide rings, of this compound having one or more epoxide function(s) with the introduction of an amine function.

In general, the mixing of the epoxide prepolymer and the curing agent is advantageously carried out under conditions that are usual for this type of method for producing epoxy resins. In particular, the formulation conditions, that is to say the type and the amount of each of the components mixed, and also the crosslinking conditions, such as the temperature, the time, etc., are conventional in themselves.

In particular, the method according to the invention preferentially comprises mixing a stoichiometric molar ratio of active hydrogen atom of the curing agent per epoxide function of the epoxide prepolymer. An active hydrogen atom is a hydrogen atom borne by a heteroatom capable of reacting with an epoxide ring, and is expressed for example, when the curing agent is of the type having an amine function, by the AHEW index (Amine H Equivalent Weight, corresponding to the weight of product per number of active H functions, expressed in g·mol⁻¹). A primary amine function R—NH₂ can react twice and can open two epoxide rings. The curing agent and the epoxide prepolymer, and also, where appropriate, additional components of the formulation containing them, are thus mixed in stoichiometric proportions (1 mol of epoxide for 1 mol of amine active H).

The calculations can be done in the following way: to produce a given weight wᵣₑₛᵢₙ of resin, with a weight w_{epoxide} of epoxide prepolymer having an EEW index (Epoxide Equivalent Weight, corresponding to a weight of product per number of epoxide functions, expressed in g·mol⁻¹) and a weight w_{curing agent} of curing agent having a given AHEW index, the following equations are applied:

$$Wepoxide = \frac{{w}_{resin}}{\frac{AHEW}{EEW} + 1}$$

$$and$$

$${w}_{curing agent} = \frac{{w}_{resin}}{\frac{EEW}{AHEW} + 1}$$

The mixture obtained can for example be poured into a mold, then treated to ensure polymerization, in particular by a heating step, for example at approximately 90° C. for a few tens of minutes, or by maintaining at ambient temperature, that is to say at a temperature approximately between 18° C. and 25° C., for a longer period of time, for example of approximately 24 h.

When the epoxide prepolymer used for producing the epoxy resin is a compound having epoxide functions according to the invention, or a mixture of such compounds, the curing agent can be any curing agent that is conventional in itself, in particular a commercially available curing agent.

The curing agent can be formed by any molecule containing at least two reactive hydrogen atoms capable of reacting with the epoxide rings so as to cause polymerization of the resin.

Conventional categories of curing agents include aliphatic, cycloaliphatic or aromatic (primary or secondary) amines and/or polyamines, acids, acid anhydrides, dicyandiamides, polysulfides, isocyanates, melamine-formaldehyde, urea-formaldehyde, phenol-formaldehyde, etc.

In particular embodiments of the invention, the crosslinking is obtained with curing agents bearing at least three active hydrogen atoms.

Conventional curing agents that can be used in the context of the invention are for example diethylenetriamine (DETA), methylenedianiline (MDA), diaminodiphenylsulfone (DDS), isophorone diamine (IPDA) or else N-aminoethylpiperazine (N-AEP), such a list being in no way limiting with respect to the invention.

The isophorone diamine, of general formula below:

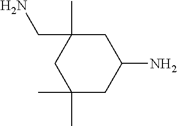

comprises two primary amine functions, that is to say four active hydrogen atoms per molecule of curing agent.

Curing agents of different types, such as sulfur-containing, anhydride, phenolic, carboxylic acid, etc., curing agents, can otherwise be used.

The curing agent can otherwise be a compound having an amine function chosen from octylamine, ethanolamine, diethylamine, piperidine, pyridine and triethylamine.

In variants of the invention, applying both in the case where the epoxide prepolymer is a compound having epoxide functions according to the invention and in the case where the epoxide prepolymer is another compound, the curing agent can be obtained by means of a prior step of modification of a compound having one or more epoxide function(s) according to the invention, or of a mixture of such compounds having one or more epoxide function(s). This modification step, carried out prior to the mixing of the curing agent with the epoxide prepolymer, consists in opening the epoxide ring(s) borne by the compound having one or more epoxide function(s) according to the invention, while simultaneously introducing into the molecule, at the level of each initial epoxide site, an amine function that will be subsequently capable of reacting with the epoxide prepolymer so as to cause crosslinking of the resin.

The curing agent of polyamine type thus obtained according to the invention, by derivatization of the epoxide functions of a compound having one or more epoxide function(s) according to the invention, comprising a polyphenol unit, has the advantage of being biobased, that is to say of being able to be obtained from renewable resources, more specifically from condensed tannins contained in biomass. It also exhibits particular characteristics which confer, on the epoxy resin that it makes it possible to obtain, advantageous mechanical properties, in particular a high rigidity and a high hardness.

In particular embodiments of the invention, the step of opening one or more epoxide ring(s) of the compound having one or more epoxide function(s) with introduction of an amine function is carried out by placing the compound having one or more epoxide function(s) in the presence of a compound, hereinafter called functionalization compound, comprising a first function capable of reacting with the epoxide ring so as to cause opening thereof, such as a thiol function, an amine function, etc., and comprising a second function being a primary amine function.

Such a functionalization compound can in particular be cysteamine, which comprises a first function which is a reactive thiol function and a second function which is a primary amine function.

In variants of the invention, the step of opening one or more epoxide ring(s) of the compound having one or more epoxide function(s) with introduction of an amine function is carried out by placing the compound having one or more epoxide function(s) in the presence of ammonia, in order to introduce onto the molecule one or more amine functions.

In particular embodiments of the method according to the invention, the compound having one or more epoxide function(s) according to the invention comprising at least two epoxide functions, the step of opening at least one epoxide ring of said compound having epoxide function(s) with the introduction of an amine function is preceded by, or carried out simultaneously with, a step of opening at least one of the epoxide rings of said compound having one or more epoxide function(s) by reaction with a nucleophilic modulation reagent, as described above, having a function capable of reacting with said epoxide ring so as to cause opening thereof, with the exclusion of an amine function, and having no other group that is reactive with respect to the epoxide functions. This step, termed modulation of the degree of crosslinking, is carried out so as to leave intact at least one epoxide ring of said compound having one or more epoxide function(s) for the step of opening at least one epoxide ring of said compound having one or more epoxide function(s) with introduction of an amine function.

In particular embodiments of the method for synthesizing an epoxy resin according to the invention, additional components can be added to the mixture of epoxide prepolymer and curing agent, in particular additives conventionally used for the production of resins of this type, such as catalysts, fillers, plasticizers, reactive diluents, stabilizers, etc.

An additional subject of the invention relates to an epoxy resin obtained by means of a production method according to the invention, corresponding to one or more of the characteristics above.

This resin is in particular of the thermosetting type.

It can be of both the epoxy-amine type and the epoxy-anhydride type. It comprises in particular units of furano-flavan type, that is to say in which a flavonoid residue is bonded by covalent bonding, at the level of the pyran ring, to a furan derivative.

The present invention also relates to the use of such an epoxy resin for obtaining materials, for example composites, intended in particular to be used for insulating electrical and/or electronic components, or else as surface coatings, in particular for metal surfaces.

This resin can otherwise be used for the production of glues or plasticizers.

The invention also relates to the use of the epoxy resin according to the invention for the manufacture of materials intended for contact with food.

According to another aspect, the present invention relates to a compound which can be used as a curing agent for the production of resins, in particular of epoxy resins, and which is capable of being obtained by means of a step of opening at least one epoxide ring of a compound having one or more epoxide function(s) according to the invention, corresponding to one or more of the characteristics above, this opening being carried out with the introduction of an amine function, or a salt thereof. This compound constitutes in particular an intermediate product of an overall method for producing an epoxy resin according to the invention, from a compound having one or more epoxide function(s) of general formula (I) above.

The step of opening the epoxide ring(s) can be carried out in accordance with one or more of the characteristics described above with reference to the method for producing the epoxy resin, in particular by means of a functionalization compound such as cysteamine, or of ammonia.

This compound can in particular correspond to general formula (VIII):

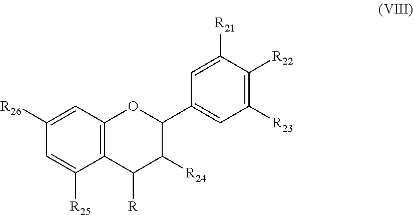

wherein:
R₂₁, R₂₂ and R₂₃, which may be identical or different, each represent a hydrogen atom, a group of formula (IX) below, or a group —OR₂₉ where R₂₉ represents a protective group for a hydroxyl function, and/or
(R₂₁ and R₂₂) together represent a group of general formula (X) or (R₂₂ and R₂₃) together represent a group of general formula (X):

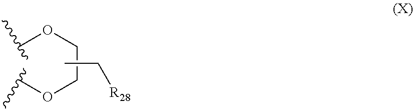

wherein R₂₈ represents a hydroxyl group, a group of formula (IX) below, or a group —OR_{29′} where R_{29′} represents a protective group for a hydroxyl function,
R₂₄ represents a hydrogen atom, a hydroxyl group, optionally protected with a protective group for a hydroxyl function, a group of formula (IX) below or a group —OR₂₇, wherein R₂₇ represents a group of general formula (XI):

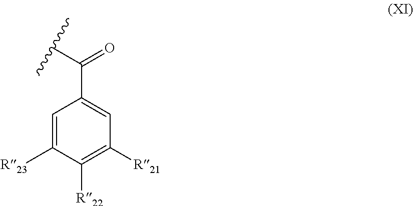

wherein R″₂₁, R″₂₂ and R″₂₃, which may be identical or different, each represent a group of formula (IX) below, or a group —OR_{29″} where R_{29″} represents a protective group for a hydroxyl function,
R₂₅ represents a hydrogen atom, a group of formula (IX) below, or a group —OR_{29′″} where R_{29′″} represents a protective group for a hydroxyl function,
R₂₆ represents a group of formula (IX) below, or a group —OR_{29′″} where R_{29′″} represents a protective group for a hydroxyl function, and
R represents a furan nucleus, which may optionally be substituted,
at least one substituent among R₂₁, R₂₂, R₂₃, R₂₄, R₂₅, R₂₆, R₂₈, R″₂₁, R″₂₂ or R″₂₃ representing a group of formula (IX) below,
said group of formula (IX) being chosen from the groups:

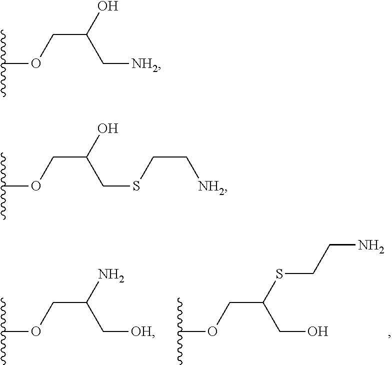

or it can be a salt thereof.

The group R can in particular correspond to the characteristics stated above in reference to the compound having one or more epoxide function(s) of general formula (I).

This compound having one or more amine function(s) can in particular correspond to general formula (VIII′):

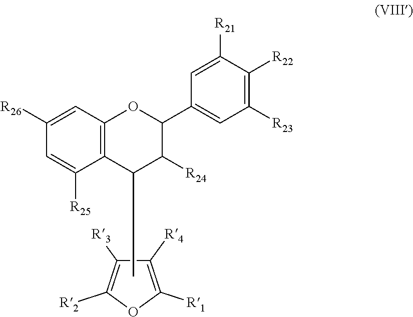

wherein R′₁, R′₂, R′₃ and R′₄ are as defined above.

This compound, which can advantageously be obtained from biomass, more specifically from condensed tannins, has numerous applications, in particular as a curing agent, for the synthesis of epoxy resins but also numerous other types of polymers, advantageously benefiting from its particular characteristics, in particular from its amine functions and from its polyaromatic unit.

General formula (VIII) encompasses all the possible combinations of isomer forms at the level of the asymmetric carbons, and all the mixtures of such isomer forms. Each particular isomer can be obtained from a mixture of isomers by means of purification methods which are conventional in themselves for those skilled in the art.

Compounds having one or more amine function(s) according to the invention can in particular correspond to general formulae (VIIIa) and (VIIIb) below:

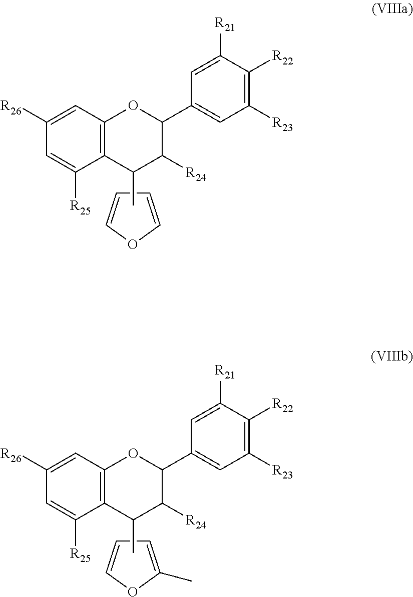

in which formulae R₂₁, R₂₂, R₂₃, R₂₄, R₂₅ and R₂₆ are as described above with reference to general formula (VIII).

In particular, in the group R, or else in general formula (VIII′), all three of R′₂, R′₃ and R′₄ can represent a hydrogen atom, and R′₁ can represent the covalent bond with the pyran ring. The compound according to the invention then corresponds to the general formula (VIIIc):

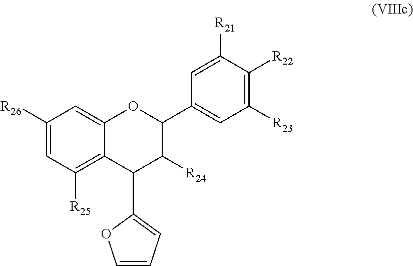

wherein R₂₁, R₂₂, R₂₃, R₂₄, R₂₅ and R₂₆ are as defined above with reference to general formula (VIII).

In the present description, the expression “furylated compound having one or more amine function(s)” will be used to designate such a compound.

In variants of the invention, R′₃ and R′₄ each represent a hydrogen atom, R′₁ represents the covalent bond with the pyran ring, and R′₂ represents a methyl radical. The compound then corresponds to general formula (VIIId) below, wherein R₂₁, R₂₂, R₂₃, R₂₄, R₂₅ and R₂₆ are as defined above with reference to general formula (VIII):

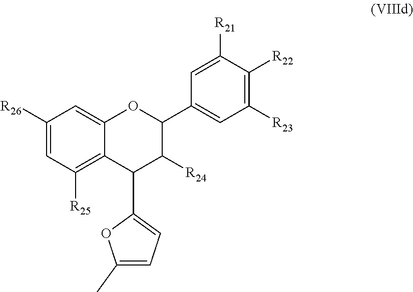

In the present description, the expression “sylvanylated compound having one or more amine function(s)” will be used to designate such a compound.

According to one particular feature of the invention, in general formula (VIII), at least two substituents, preferably at least three substituents, and preferably at least four substituents, among R₂₁, R₂₂, R₂₃, R₂₄, R₂₅, R₂₆, R₂₈, R″₂₁, R″₂₂ and R″₂₃, represent a group of formula (IX). By way of example, two, or three, or four, or five, or six substituents, among R₂₁, R₂₂, R₂₃, R₂₄, R₂₅, R₂₆, R₂₈, R″₂₁, R″₂₂ and R″₂₃, represent a group of formula (IX).

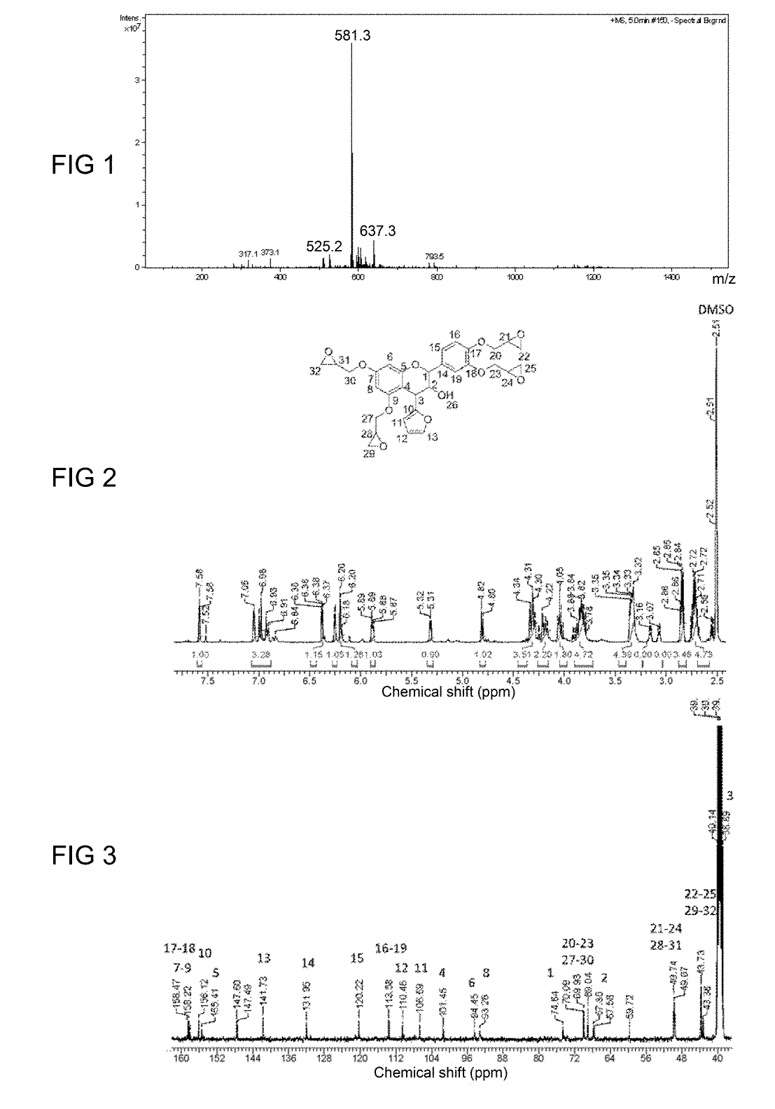

The features and advantages of the invention will emerge more clearly in light of the implementation examples below, provided simply by way of illustration and which are in no way limiting with respect to the invention, with the support of FIGS. 1 to 9b, wherein:

FIG. 1 shows the MS(+) mass spectrum of a compound having epoxide functions according to the invention, synthesized by epoxidation of a compound obtained by depolymerization of condensed tannins from white seeds using furan;

FIG. 2 shows the ¹H NMR spectrum of the compound of FIG. 1;

FIG. 3 shows the ¹³C NMR spectrum of the compound of FIG. 1;

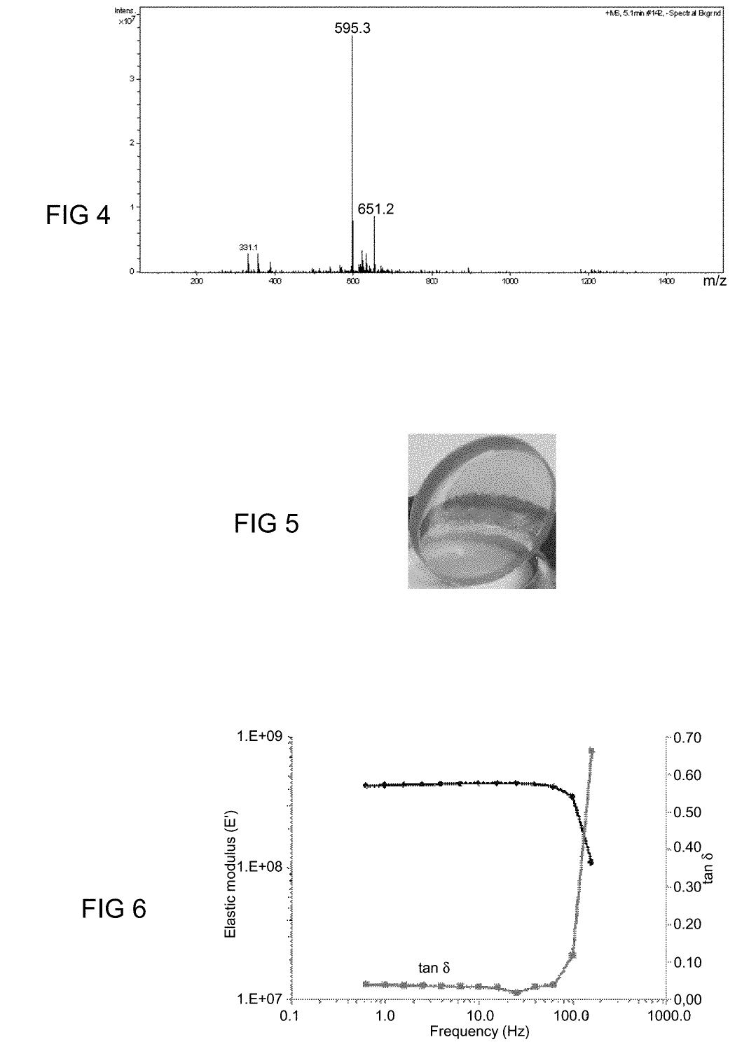

FIG. 4 shows the MS(+) mass spectrum of a compound having epoxide functions according to the invention, synthesized by epoxidation of a compound obtained by depolymerization of condensed tannins from white seeds using sylvan;

FIG. 5 shows a photograph of a sample of epoxy resin formulated from a compound having epoxide functions according to the invention as epoxide prepolymer and from isophorone diamine as curing agent;

FIG. 6 represents curves illustrating the change in the elastic modulus and in the tan δ parameter as a function of the stress frequency, of a test sample of an epoxy resin formulated from a compound having epoxide functions according to the invention as epoxide prepolymer and from isophorone diamine as curing agent;

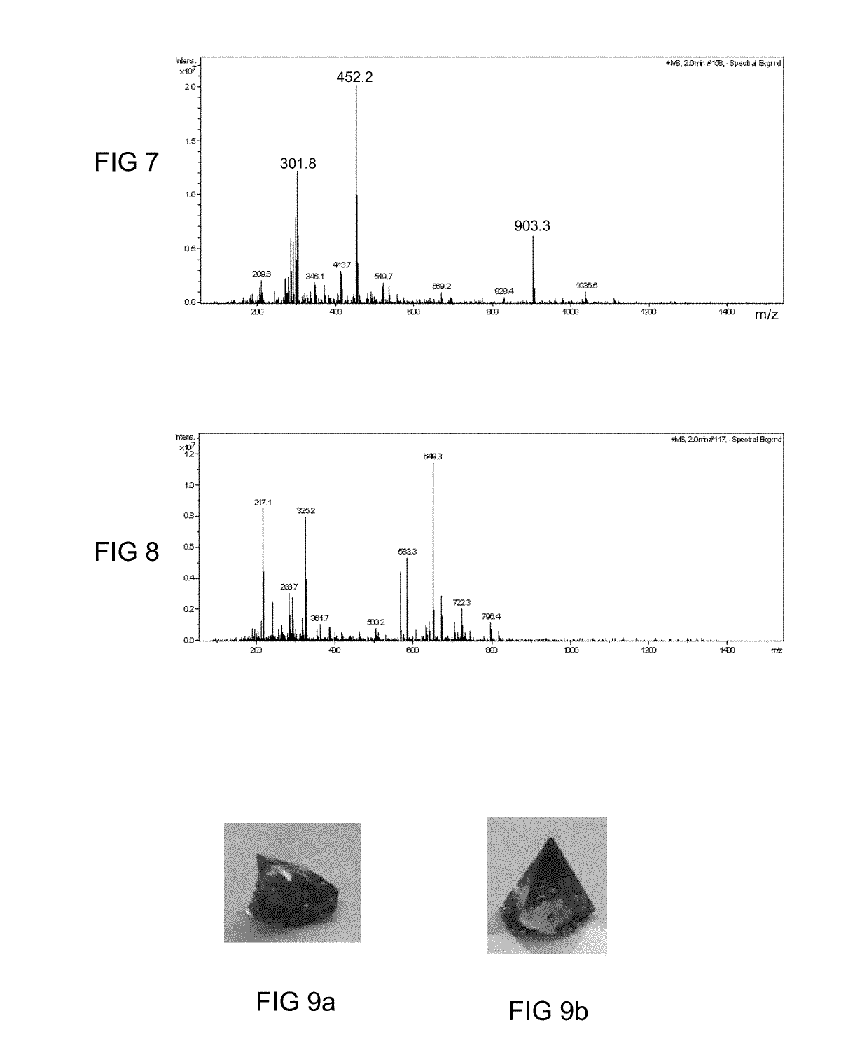

FIG. 7 shows the MS(+) mass spectrum of a compound having amine functions according to the invention, synthesized by derivatization by addition of cysteamine of a compound having one or more epoxide function(s) according to the invention, itself synthesized by epoxidation of a compound obtained by depolymerization of condensed tannins from white seeds using sylvan;

FIG. 8 shows the MS(+) mass spectrum of a compound having amine functions according to the invention, synthesized by derivatization by addition of ammonia of a compound having one or more epoxide function(s) according to the invention, itself synthesized by epoxidation of a compound obtained by depolymerization of condensed tannins from white seeds using sylvan; and

FIGS. 9a and 9b show images of an epoxy resin in accordance with the invention obtained, respectively, after deformation (1 min at 90° C., then cooling under deforming stress) for FIG. 9a, and after heating (1 min at 90° C., then return to ambient temperature without stress), for FIG. 9b.

### A. SYNTHESIS OF COMPOUNDS HAVING EPOXIDE FUNCTIONS IN ACCORDANCE WITH THE INVENTION

Compounds having epoxide functions in accordance with the invention are prepared by glycidylation of the products of depolymerization of condensed tannins either using furan (of formula (VIIa) above), or using sylvan (of formula (VIIb) above). The condensed tannins are used in the form of industrial seed extracts produced from marcs originating from white wine productions (“white seed tannins”) (high quality grade), obtained from the Union des Distilleries de la Meditérranée [Mediterranean Union of Distilleries].

A.1. Depolymerization of Condensed Tannins Using Furan

The white seed tannin extract (5.0 g) is dissolved in MeOH (200 ml), in a bottle, then furan (108 ml) is added, followed by hydrochloric methanol (83 ml of fuming HCl in 108 ml of MeOH) without stirring. The mixture is brought to 40° C., for 30 min, and then cooled to 0° C. 500 ml of an aqueous Na₂CO₃ solution (106 g·l⁻¹) are then added. The mixture is extracted with ethyl acetate (EtOAc) (3×400 ml), then subjected to evaporation. A pasty brown-black solid (3.34 g) is obtained, which is taken up in diethyl ether (Et₂O) (200 ml), triturated and sonicated, then washed with brine (300 ml). These operations are repeated twice, and the solutions obtained are dried (Na₂SO₄) and then evaporated so as to obtain a brownish pasty solid (2.40 g), which consists of a mixture of furylated extension units and terminal units below:
furylated extension units corresponding to general formula (V) above:

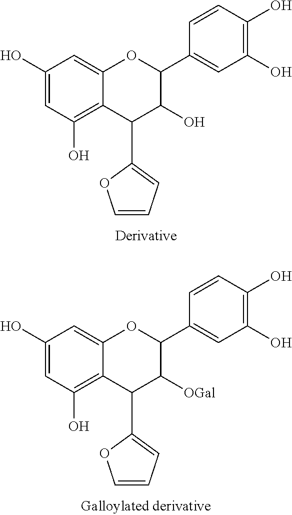

terminal units:

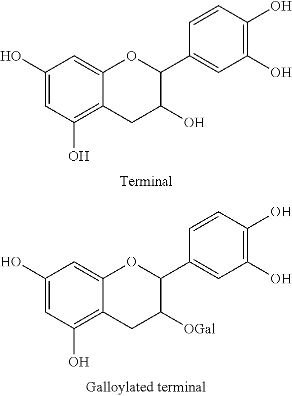

where Gal represents a gallate group of formula (VI) defined above.

A.2. Glycidylation of the Products Obtained by Depolymerization of Condensed Tannins Using Furan (A.1)

The products derived from the depolymerization of the tannins with furan (2.40 g) are dissolved in epichlorohydrin (20 ml), and benzyltriethylammonium chloride is added (154 mg). The reaction continues for 1 h at 100° C. An aqueous sodium hydroxide solution (aqueous NaOH, 20% by weight) is added (27 ml), as is additional benzyltriethylammonium chloride (308 mg). The reaction continues for 1 h 30 at 30° C.

The product is then extracted with ethyl acetate (3×50 ml), dried and evaporated. A yellow-orange oil is obtained which is triturated from hexane so as to remove the remaining traces of epichlorohydrin. The oil obtained is then deposited on silica gel and eluted with pure ethyl acetate, to give, after evaporation, a yellow oil (2.77 g), referred to as Ef.

This oil contains a mixture of compounds having epoxide functions of formula (Ic) below:

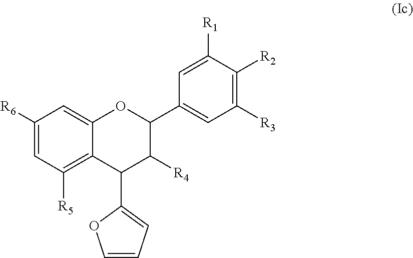

The compounds which go to making up this oil are characterized by ultra-high performance liquid chromatography-mass spectrometry (UPLC-MS) analysis. This analysis consists in performing a separation of the depolymerization reaction products by ultra performance liquid chromatography (Waters UPLC system) coupled in series to a diode array detector (DAD) and to a mass spectrometer (Brucker AmaZonX model) (UPLC-MS). The synthesis samples are analyzed extemporaneously, the product being diluted where appropriate for a final concentration of 1 g·l⁻¹. The samples (2 μl) are injected onto a Waters Acquity Atlantis HSS T3 1.8 μm-2.1×100 mm column, and eluted with the solvents A (99:1 H₂O:HCOOH) and B (19:1:80 H₂O:HCOOH:MeCN) according to the A/B gradient: 99% to 1% linear, 8 min; 1% isocratic, 1 min; 1% to 99% linear, 1 min; for a flow rate of 550 μl·min⁻¹. The UV chromatogram recorded at 280 nm allows phenolic derivatives to be analyzed. The MS(+) chromatogram allows the products to be identified on the basis of the m/z values.

The compounds having epoxide functions of catechin-furan type, corresponding to general formula (Ii):

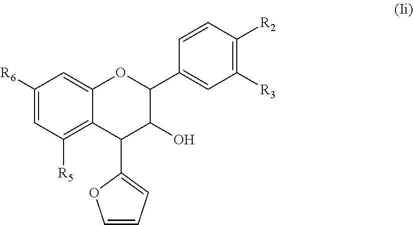

are purified by chromatography for formal characterization by ¹H NMR and ¹³C NMR spectrometry and by mass spectrometry.

The purification is carried out by flash chromatography on an Interchim PF430 instrument and by solid deposit on 15 μm silica gel, with a 0→100% EtOAc/heptane gradient. The fractions of interest are collected and evaporated to dryness under vacuum.

The acquisition of the NMR spectra is carried out on spectrometers at 400 MHz and 600 MHz (Brucker). The samples are dissolved in DMSO-d6. The spectra are obtained at 25° C. and the chemical shifts are given in parts per millions (ppm). The assignment of the signals (protons and carbons) is done by choosing as internal reference the chemical shifts of the DMSO-d6, that is to say 2.5 ppm for ¹H and 39.5 ppm for ¹³C. Other than the 1D experiments (¹H and ¹³C), the interpretations of structures are carried out by means of HSQC and HMBC two-dimensional NMR experiments.

By way of example, the spectra obtained for the particular compound of general formula (Ii′):

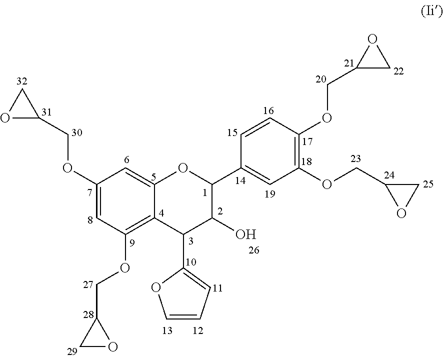

are shown, respectively, in FIG. 1 for the mass spectrum (M+H⁺)/z=581, in FIG. 2 for the ¹H NMR spectrum and in FIG. 3 for the ¹³C NMR spectrum (in DMSO-d6). These spectra confirm the structure of the compound (Ii′) in accordance with the invention above, as shown in Table 1 below which summarizes the NMR data obtained.

TABLE 1
NMR characterization of the compound having epoxide functions
(Ii′) in accordance with the invention - m, d and dd signify
respectively multiplet, doublet and doublet of doublets.

| Assignment | ¹³C (ppm) | Type | ¹H (ppm) | Coupling J |
|---|---|---|---|---|
| 1 | 74.6 | CH | 4.80 | d - 7.3 Hz |
| 2 | 67.8 | CH | 4.05 | m |
| 3 | 39.1 | CH | 4.20 | m |
| 4 | 101.4 | Q | — | |
| 5 | 155.4 | Q | — | |
| 6 | 94.4 | CH | 6.19 | d - 2.2 Hz |
| 7-9 | 158.5-158.2-158.1 | Q | — | |
| 17-18 | | | | |
| 8 | 93.3 | CH | 6.24 | d - 2.2 Hz |
| 10 | 156.1 | Q | — | |
| 11 | 106.7 | CH | 5.88 | d - 3.1 Hz |
| 12 | 110.5 | CH | 6.37 | dd - 1.8/3.1 Hz |
| 13 | 141.7 | CH | 7.58 | d - 1.8 Hz |
| 14 | 132.0 | Q | — | |
| 15 | 120.2 | CH | 6.91 | dd - 2.0/8.4 Hz |
| 16 | 113.6 | CH | 6.98 | d - 8.4 Hz |
| 19 | 113.6 | CH | 7.04 | d - 2.0 Hz |
| 20-23 | 70.1-69.9 | CH₂ | 4.30-3.83 | m |
| 27-30 | | | | |
| 21-24 | 49.8 | CH | 3.33 | m |
| 28-31 | | | | |
| 22-25 | 43.7 | CH₂ | 2.72 | m |
| 29-32 | 43.4 | | 2.84 | |
| 26 | | OH | 5.31 | d - 4.6 Hz |

The spectra obtained for the particular compound of general formula (Ii″):

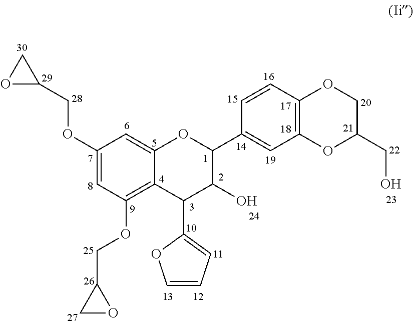

make it possible to obtain the NMR data indicated in Table 2 below.

TABLE 2
NMR characterization of the compound having epoxide functions
(Ii″) in accordance with the invention - m, d and dd signify
respectively multiplet, doublet and double of doublets.

| Assignment | ¹³C (ppm) | Type | ¹H (ppm) | Coupling J |
|---|---|---|---|---|
| 1 | 74.5 | CH | 4.74 | m |
| 2 | 67.9 | CH | 4.00 | m |
| 3 | 39.2 | CH | 4.17 | m |
| 4 | 101.4 | Q | — | |
| 5 | 155.5 | Q | — | |
| 6 | 94.4 | CH | 6.17 | d - 2.1 Hz |
| 7-9 | 158.5 | Q | — | |
| 8 | 93.2 | CH | 6.22 | d - 2.1 Hz |
| 10 | 156.1 | Q | — | |
| 11 | 106.7 | CH | 5.86 | d - 3.0 Hz |
| 12 | 110.4 | CH | 6.35 | dd - 2.0/3.0 Hz |
| 13 | 141.7 | CH | 7.55 | d - 2.0 Hz |
| 14 | 132.0 | Q | — | |
| 15 | 119.5 | CH | 6.75 | m |
| 16 | 116.3 | CH | 6.81 | m |
| 17-18 | 142.5 | Q | — | |
| 19 | 116.0 | CH | 6.91 | m |
| 20 | 65.2 | CH₂ | 4.30-3.97 | m |
| 21 | 73.7 | CH | 4.13 | m |
| 22 | 59.9 | CH₂ | 3.61 | m |
| 23 | | OH | 5.03 | m |
| 24 | | OH | 5.29 | m |
| 25-28 | 69.0 | CH₂ | 4.29-3.79 | m |
| 26-29 | 49.6 | CH | 3.03 | m |
| 27-30 | 43.4 | CH₂ | 2.72-2.53 | dd - 4.2/5.1 Hz + dd - |
| | 43.7 | | 2.83-2.68 | 2.5/5.1 Hz |
| | | | | dd - 4.2/5.1 Hz + m |

These data confirm the structure of the compound (Ii″) in accordance with the invention above.

A.3. Depolymerization of Condensed Tannins Using Sylvan

In a round-bottomed flask, the white seed tannin extract (8.0 g) is dissolved in MeOH (300 ml), then the sylvan (100 ml) followed gently by the fuming HCl (3.33 ml) are added, with stirring. The mixture is brought to 30° C. for 60 min. 400 ml of an aqueous Na₂CO₃ solution are added (5.3 g·l⁻¹), then an extraction with EtOAc (3×400 ml) is carried out. The solution is evaporated to give a pasty brownish solid (5.7 g), which is taken up with Et₂O (200 ml), triturated and sonicated, then washed with brine (300 ml). These operations are repeated twice, and the resulting solutions are combined, and dried with Na₂SO₄. The solution is evaporated so as to obtain a beige bullate solid (2.40 g), consisting of a mixture of sylvanylated extension units and terminal units below:
sylvanylated extension units corresponding to general formula (V) above:

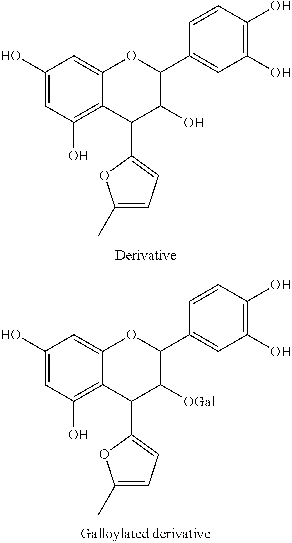

terminal units:

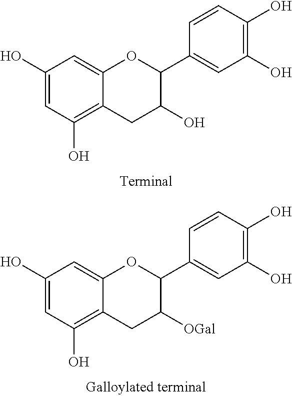

where Gal represents a gallate group of formula (VI) defined above.

A.4. Glycidylation of the Products Obtained by Depolymerization of Condensed Tannins Using Sylvan (A.3)

The products resulting from the depolymerization of the tannins using sylvan (9.00 g) are dissolved in epichlorohydrin (98 ml), and benzyltriethylammonium chloride is added (707 mg). The reaction continues for 1 h at 100° C. In a second step, an aqueous sodium hydroxide solution (aqueous NaOH, 20% by weight) is added (124 ml), as is additional benzyltriethylammonium chloride (1.41 g). The reaction continues for 1 h 30 at 30° C. The product is then extracted with ethyl acetate (3×200 ml), dried and evaporated. A brown oil is obtained which is triturated from hexane in order to remove the remaining traces of epichlorohydrin. The oil obtained is then deposited on silica gel and eluted with pure ethyl acetate to give, after evaporation, an orange oil (16.0 g), referred to as Es.

This oil contains a mixture of compounds having epoxide functions of formula (Id) below:

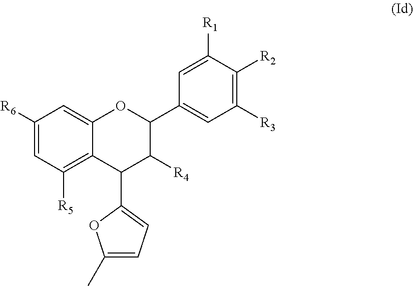

The compounds which go to make up this oil are characterized by ultra-high performance liquid chromatography-mass spectrometry (UPLC-MS) analysis, as described above.

The compounds having epoxide functions of catechin-sylvan type, correspond to general formula (Ij):

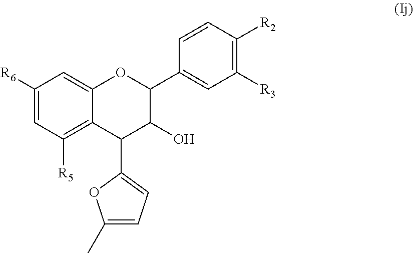

are purified for formal characterization by ¹H NMR and ¹³C NMR spectrometry and by mass spectrometry, as described above.

By way of example, the mass spectrum (M+H⁺)/z=595 obtained for the particular compound of general formula (Ij′):

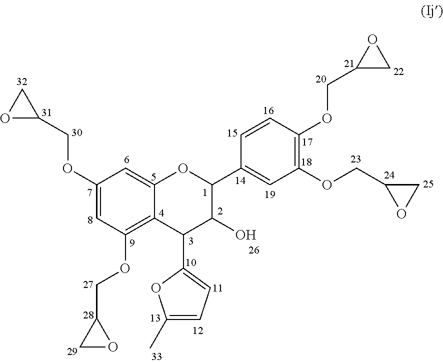

is show in FIG. 4.

The ¹H NMR and ¹³C NMR spectra also carried out make it possible to obtain NMR data indicated in Table 3 below.

TABLE 3
NMR characterization of the compound having epoxide functions
(Ij′) in accordance with the invention - m, d and bs signify
respectively multiplet, doublet and broad singlet.

| Assignment | ¹³C (ppm) | type | ¹H (ppm) | Coupling J |
|---|---|---|---|---|
| 1 | 74.6 | CH | 4.83 | d - 8.8 Hz |
| 2 | 67.9 | CH | 4.03 | m |
| 3 | 38.6 | CH | 4.11 | m |
| 4 | 101.6 | Q | — | — |
| 5 | 155.5 | Q | — | — |
| 6 | 94.5 | CH | 6.17 | m |
| 7 & 9 | 158.5 | Q | — | — |
| | 158.1 | | | |
| 8 | 93.2 | CH | 6.23 | m |
| 10 | 154.6 | Q | — | — |
| 11 | 107.4 | CH | 5.70 | m |
| 12 | 106.5 | CH | 5.94 | m |
| 13 | 150.1 | CH | — | — |
| 14 | 132.1 | Q | — | |
| 15 | 120.3 | CH | 6.92 | m |
| 16 | 113.7 | CH | 6.98 | d - 8.3 Hz |
| 17 & 18 | 147.7 | Q | — | — |
| | 147.6 | | | |
| 19 | 113.7 | CH | 7.05 | m |
| 20-23 | 70.1-69.9 | CH₂ | 4.30-3.84 | m |
| 27-30 | | | | |
| 21-24 | 49.9-49.7 | CH | 3.32 | m |
| 28-31 | | | | |
| 22-25 | 43.7-43.4 | CH₂ | 2.83-2.70 | m |
| 29-32 | | | | |
| 26 | — | OH | 5.27 | bs |
| 33 | 13.1 | CH₃ | 2.24 | m |

These data, and also the mass spectrum obtained, confirm the structure of the compound (Ij′) in accordance with the invention above.

A.5. Assaying of the Epoxide Functions of the Compounds Synthesized

The assaying of the epoxide functions makes it possible to obtain the EEW epoxide index.

Principle of the Assay

The objective of the assay is to open the epoxide ring under acid conditions and to determine the amount of acid having reacted, and therefore the amount of epoxide functions present.

The product to be analyzed is weighed exactly (approximately 100 mg) and dissolved in 13 ml of a 0.2 M solution of HCl in pyridine. The reaction medium is then heated at 120° C. for 20 min and then brought back to ambient temperature. The colorimetric assay of the excess acid is carried out with an exactly titrated solution of sodium hydroxide in methanol (approximately 6 to 7 mmol·l⁻¹), in the presence of phenolphthalein. The assay is carried out in triplicate for each product. The 0.2 M HCl solution is also assayed with sodium hydroxide (blank).

The epoxide index is calculated according to the following equation, for a given weight of prepolymer w_{epoxide}, a known sodium hydroxide concentration C_{NaoH} and volumes of sodium hydroxide V₀ for the blank (acid without epoxide) and V_{epoxide} for the excess acid after reaction with the prepolymer:

$$EEW = \frac{{w}_{epoxide}}{( {V}_{0} - {V}_{epoxide} ) \cdot {C}_{NaOH}}$$

Experimental Results

DGEBA (Diglycidyl Ether of Bisphenol A) Reference Assay

Theoretical value (calculated): the molecular weight of DGEBA is 340 g·mol⁻¹ and the structure contains two epoxide functions, that is to say an EEWₜₕ equal to 170 g·mol⁻¹ of epoxide.

Experimental value (obtained by assaying): EEW=165 g·mol⁻¹ of epoxide, that is to say a difference of 3% with the theoretical value, confirming the reliability of the assay method.
Assaying of the oil Ef (products resulting from the glycidylation of the industrial extract of grape seed tannins depolymerized in the presence of furan):

EEWₑₓₚ=116 g·mol⁻¹
Assaying of the oil Es (products resulting from the glycidylation of the industrial extract of grape seed tannins depolymerized in the presence of sylvan):

EEWₑₓₚ=133 g·mol⁻¹

The values of these EEW indices are lower than that of DGEBA and conform to the UPLC-MS analyses indicating a high degree of glycidylation.

### B. METHOD FOR PRODUCING EPOXY RESINS IN ACCORDANCE WITH THE INVENTION

A method for producing an epoxy resin in accordance with the invention involves the mixing of a compound having one or more epoxide function(s) according to the invention, as epoxide prepolymer, with a curing agent of polyamine type, more specifically with a stoichiometric molar ratio of amine active H per epoxide function.

The curing agent used is isophorone diamine, which comprises two primary amine functions, that is to say 4 active H per mole of curing agent.

B.1. Starting from the Oil Ef

The oil Ef obtained by oxidation of the reaction crude of the depolymerization of condensed tannins using furan (EEW=116 g·l⁻¹) is used to produce an epoxy resin, as follows.

950 mg of oil Ef and 372 μl of isophorone diamine are mixed so as to prepare a homogeneous mixture. The mixture is cast into a test specimen and heated at 90° C. for 20 min. A disk of hard yellow translucent resin is obtained, an image of which is shown in FIG. 5.

B.2. Starting from the Oil Es

The oil Es obtained by epoxidation of the reaction crude of the depolymerization of condensed tannins using sylvane (EEW=133 g·l⁻¹) is used to produce an epoxy resin, as follows.

1.1 g of oil Ef and 377 μl of isophorone diamine are mixed so as to produce a homogeneous mixture. The mixture is cast into a test specimen and heated at 90° C. for 30 min under pressure (200 bar). A test specimen 6.24 mm long, 5.28 mm wide and 0.98 mm thick, of hard yellow translucent resin is obtained. The appearance of this resin is similar to that shown in FIG. 5.

A study of the mechanical strength of this epoxy resin was carried out by dynamic mechanical analysis (DMA) at 25° C. of the resin test specimen obtained, by means of a TA Instruments DMA 2980 analyzer. The DMA measurement conditions are the following: scan of 0.6 to 300 Hz for a deformation of 0.05 mm, at 25° C. The curves obtained, representing the elastic modulus and the tan δ factor as a function of the stress frequency, are shown in FIG. 6. It is deduced therefrom that the characteristics of the epoxy resin according to the invention are those of a hard material, with a tan δ damping factor of 3×10⁻², that is to say in the range of those of Plexiglas®, of concrete and of brick, and with an elastic modulus of 0.43 GPa.

### C. PRODUCTION OF POLYAMINE CURING AGENTS IN ACCORDANCE WITH THE INVENTION

Polyamine curing agents in accordance with the invention are produced from, respectively, the oils Ef and Es obtained as indicated above, according to the protocols below.

C.1. Derivatization by Addition of Cysteamine

1.0 g of oil (Ef or Es) is dissolved in MeOH (50 ml). Cysteamine hydrochloride is added (3.8 g), followed by N,N-diisopropylethylamine (2.9 ml). The reaction is left to take place for 20 h at ambient temperature. Sodium carbonate is added (8.3 g), and then the MeOH is evaporated off. The product is extracted by trituration of the residue from acetone (3×50 ml). The three organic phases are combined, dried, and evaporated to give an orangey oil.

Starting from the oil Es, a mixture of compounds of general formula (VIIIc) below is obtained, and starting from the oil Ef, a mixture of compounds of general formula (VIIId) above is obtained, with, for these two formulae, a group (IX) which can correspond to the formulae:

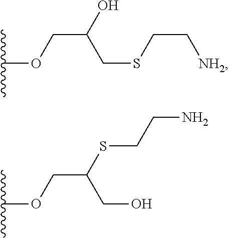

Compounds having particular amine functions thus obtained are derivatives of catechin, and correspond to general formulae (VIIIe) and (VIIIf) below:

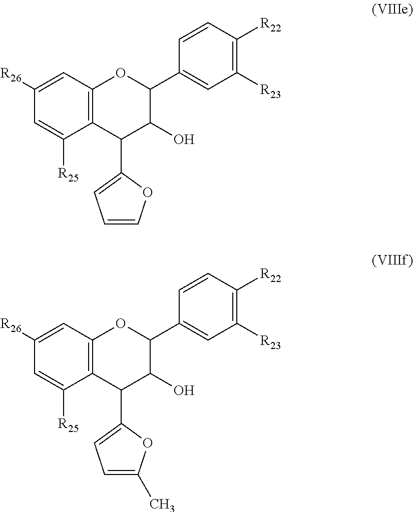

wherein the group of formula (IX) can correspond to the formulae:

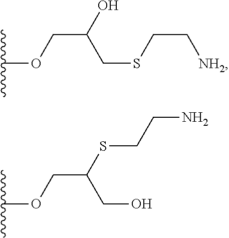 [SMILES: *OCC(O)CSCCN ; *OCC(CO)SCCN]

Compounds of the invention thus obtained, which are particularly advantageous for use as curing agents, since they bear four primary amine functions, each being able to react with two epoxide functions, correspond to general formula (VIIIg):

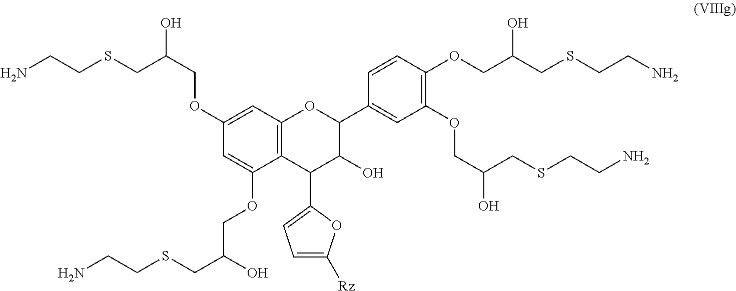

wherein R_{z} represents a hydrogen atom or a methyl radical.

By way of example, FIG. 7 shows the mass spectrum obtained for the compound according to the invention of general formula (VIIIg) above, wherein Rz represents a methyl radical: (M+H⁺)/z=903 with z=1, (M+2H⁺)/z=452 with z=2, (M+3H⁺)/z=302 with z=3.

C.2. Derivatization by Addition of Aqueous Ammonia

In a pressure-resistant threaded flask, the oil (1.0 g) is dissolved in isopropanol (iPrOH) (67 ml). Aqueous ammonia (aqueous NH₃, 25%) is added (33 ml). The tube is hermetically stoppered with a stopper which has a PTFE septum, and brought to 85° C. with stirring for 6 h. The aqueous ammonia and the iPrOH are then evaporated to dryness so as to directly give the expected product in the form of a viscous yellow-orange oil (1.2 g; quantitative yield).

Starting from the oil Es, a mixture of compounds of general formula (VIIIc) above is obtained, and starting from the oil Ef, a mixture of compounds of general formula (VIIId) above is obtained, with, for these two formulae, a group (IX) which can correspond to the formulae:

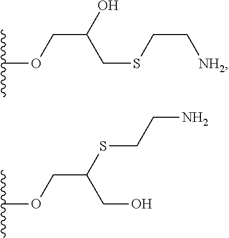

Particular compounds having amine functions thus obtained are catechin derivatives, and correspond to general formulae (VIIIk) and (VIIIm) below:

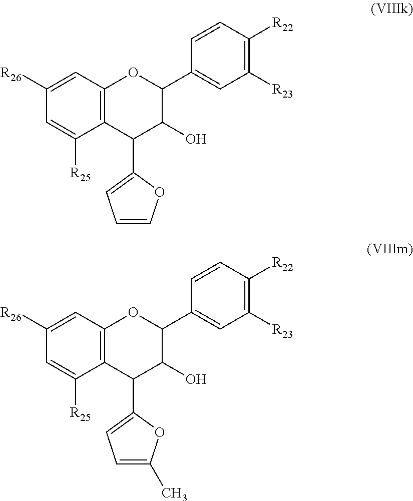

wherein the group of formula (IX) can correspond to the formulae:

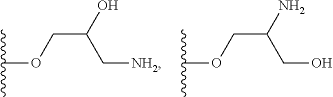 [SMILES: *OCC(O)CN ; *OCC(N)CO]

Compounds of the invention thus obtained that are particularly advantageous for use as curing agents, since they bear four primary amine functions, each being able to react with two epoxide functions, correspond to general formula (VIIIn):

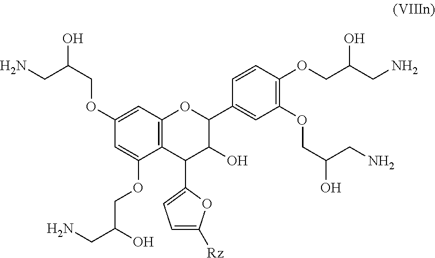

wherein R_{z} represents a hydrogen atom or a methyl radical.

The structures of all of the compounds having one or more amine function(s) according to the invention that are obtained as indicated above were confirmed by mass spectrometry and NMR spectrometry analysis. By way of example, FIG. 8 shows the mass spectrum obtained for the compound according to the invention of general formula (VIIIn) above, wherein Rz is a hydrogen atom: (M+H⁺)/z=649 with z=1, (M+2H⁺)/z=325 with z=2, (M+3H⁺)/z=217 with z=3.

The NMR spectra obtained for the compound of general formula (VIIIo) according to the invention below:

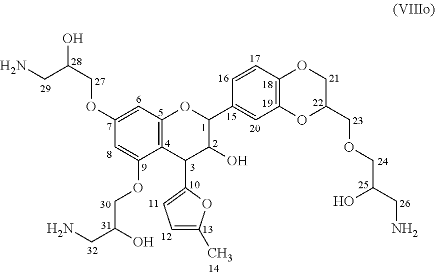

make it possible to obtain the NMR data indicated in Table 4 below.

TABLE 4
NMR characterization of the compound having epoxide functions
(VIIIo) in accordance with the invention - m, d and s signify
respectively multiplet, doublet and singlet

| Assignment | ¹³C (ppm) | Type | ¹H (ppm) | Coupling J |
|---|---|---|---|---|
| 1 | 74.1 | CH | 4.79 | m |
| 2 | 67.7 | CH | 3.97 | m |
| 3 | 38.5 | CH | 4.11 | m |
| 4 | 101.0 | Q | — | — |
| 5 | 155.2 | Q | — | — |
| 6 | 93.8 | CH | 6.11 | d - 2.6 Hz |
| 7 | 158.7 | Q | — | — |
| 8 | 92.4 | CH | 6.15 | d - 2.6 Hz |
| 9 | 158.3 | Q | — | — |
| 10 | 154.5 | Q | — | — |
| 11 | 105.9 | CH | 5.92 | — |
| 12 | 106.9 | CH | 5.69 | — |
| 13 | 149.7 | Q | — | — |
| 14 | 13.1 | CH₃ | 2.23 | s |
| 15 | 132.0 | Q | — | — |
| 16 | 119.4 | CH | 6.78 | m |
| 17 | 115.9 | CH | 6.83 | m |
| 18 | 142.2 | Q | — | |
| 19 | 142.2 | Q | — | m |
| 20 | 115.5 | CH | 6.94 | m |
| 21 | 64.7 | CH₂ | 4.31-4.01 | m |
| 22 | 71.7 | CH | 4.31 | m |
| 23 | 69.0 | CH₂ | 3.65 | m |
| 24 | 73.4 | CH₂ | 3.42-3.39 | m |
| 25 | 70.3 | CH | 3.52 | m |
| 26 | 44.4 | CH₂ | 2.59-2.46 | m |
| 27 | 70.0 | CH₂ | 3.92-3.83 | m |
| 28 | 70.0 | CH | 3.71 | m |
| 29 | 44.5 | CH₂ | 2.69-2.58 | m |
| 30 | 69.5 | CH₂ | 3.81-3.71 | m |
| 31 | 69.9 | CH | 3.59 | m |
| 32 | 44.3 | CH₂ | 2.50-2.35 | m |

These data confirm the structure of the compound (VIIIo) in accordance with the invention above.

### D. EXAMPLES OF SYNTHESIS OF EPOXY RESINS IN ACCORDANCE WITH THE INVENTION

Epoxy resins are produced according to various methods in accordance with the present invention, in the following way.

D.1 Synthesis of a Material from the Prepolymer Es with the Octylamine Curing Agent

142 mg of curing agent are dissolved in 222 mg of oil Es. The mixture is brought to 90° C. for 60 min. A hard translucent resin is obtained.

D.2. Synthesis of a Material from the Prepolymer Es with the Diethylamine Curing Agent

103 mg of curing agent are dissolved in 103 mg of oil Es. The mixture is brought to 90° C. for 20 min. A shape-memory translucent resin is obtained.

D.3. Synthesis of a Material from the Prepolymer Es with Piperidine

151 mg of piperidine are dissolved in 230 mg of oil Es. The mixture is brought to 90° C. for 20 min. A resin with thermoplastic properties, which becomes liquid again under hot conditions (90° C.), is obtained.

D.4. Synthesis of a Material from the Prepolymer Es with the Triethylamine Curing Agent

108 mg of curing agent are dissolved in 137 mg of oil Es. The mixture is brought to 90° C. for 20 min. A hard translucent resin is obtained.

D.5. Synthesis of a Material from the Prepolymer Es with the Pyridine Curing Agent

113 mg of curing agent are dissolved in 183 mg of oil Es. The mixture is brought to 90° C. for 20 min. A hard opaque resin which is very dark red in color is obtained.

D.6. Synthesis of a Material from the Prepolymer Es with the Ethanolamine Curing Agent

71 mg of curing agent are dissolved in 299 mg of oil Es. The mixture is brought to 90° C. for 20 min. A shape-memory resin is obtained.

D.7. Synthesis of a Material from the Prepolymer Es with the Pyrrolidine Curing Agent

72 mg of curing agent are dissolved in 150 mg of oil Es. The mixture is brought to 90° C. for 20 min. A hard resin is obtained.

D.8. Synthesis of a Material from the Commercial Prepolymer DGEBA with the Curing Agent-Sylv-NH₃ (Obtained as Described in C.2.)

99 mg of curing agent are dissolved in 99 mg of ethylene glycol. 120 mg of DGEBA are added. The mixture is brought to 90° C. for 20 min. A hard translucent resin is obtained.

D.9. Synthesis of a Material from the Prepolymer (Es) with the Curing Agent Obtained by Derivatization, by Addition of Aqueous Ammonia, of the Oil Ef (as Described in C.2.)

98 mg of curing agent are dissolved in 98 mg of ethylene glycol. 118 mg of oil Es are added. The mixture is brought to 90° C. for 20 min. A hard resin is obtained.

D.10. Synthesis of a Material from the Prepolymer (Es) with the Curing Agent Obtained by Derivatization, by Addition of Aqueous Ammonia, of the Oil Es (as Described in C.2/)

101 mg of curing agent are dissolved in 101 mg of ethylene glycol. 94 mg of oil Es are added. The mixture is brought to 90° C. for 20 min. A hard translucent resin is obtained.

D.11. Synthesis of a Material from the Prepolymer (Es) with the Curing Agent Obtained by Derivatization, by Addition of Cysteamine, of the Oil Es (as Described in C.1/).

93 mg of curing agent are dissolved in 93 mg of ethylene glycol. 115 mg of oil Es are added. The mixture is brought to 90° C. for 20 min. A hard resin is obtained.

D.12. Synthesis of a Material from the Prepolymer (Es) with, as Curing Agent, the Polyphenols Resulting from the Depolymerization of Tannins with Sylvan (Obtained as Described in A.3/)

56 mg of curing agent are dissolved in 123 mg of oil Es. The mixture is brought to 90° C. for 20 min. A hard translucent resin is obtained.

D.13. Synthesis of a Material from the Prepolymer (Es) with, as Curing Agent, a Furan Derivative, Furfurylamine

3.52 g of curing agent are dissolved in 1.28 g of oil Es. The mixture is left at ambient temperature for 16 h, and then brought to 90° C. for 20 min. A resin which has viscoelastic properties is obtained: it is possible to push the nails into said resin, and the marks left by said nails disappear in a few instants.

### E. SYNTHESIS OF SHAPE-MEMORY RESINS IN ACCORDANCE WITH THE INVENTION

The prepolymer Es (1 g) is mixed with diethylamine (1 g).

The mixture is poured into a pyramidal silicone mold, and cured at 90° C. for 1 h, and then the mold is left to cool to ambient temperature.

After removal from the mold, a hard object is obtained. When the object is reheated at 90° C., it becomes soft, and it is possible to force it to adopt another shape, which is preserved if the object is cooled to ambient temperature. If the object is again brought to 90° C., it softens again, and adopts the shape that it had at the time of the initial molding.

FIGS. 9a and 9b illustrate, respectively, the resin obtained: after deformation (1 min at 90° C., then cooling under deforming stress), demonstrating the maintaining of the deformation applied; and after heating (1 min at 90° C., then return to ambient temperature without stress), demonstrating the return to the initial pyramidal shape.

1. A compound having one or more epoxide function(s) of general formula (I): 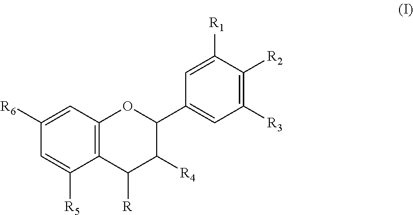
wherein:
R₁, R₂ and R₃, which is identical or different, each represent a hydrogen atom, a group of formula (III), or a group —OR₉ where R₉ represents a protective group for a hydroxyl function: 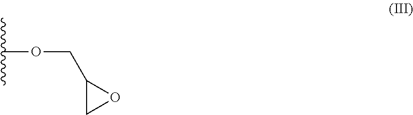 [SMILES: *OCC1CO1]
and (R₁ and R₂) or (R₂ and R₃) together represent a group of general formula (IV): 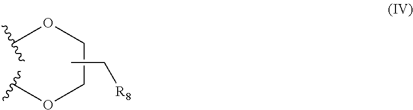
wherein R₈ represents a hydroxyl group, a group of formula (III), or a group —OR_{9′} where R_{9′} represents a protective group for a hydroxyl function,
R₄ represents a hydrogen atom, a hydroxyl group, optionally protected with a protective group, a group of formula (III) or a group —OR₇, wherein R₇ represents a group of general formula (II): 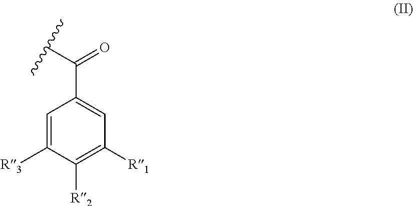
wherein R″₁, R″₂ and R″₃, which is identical or different, each represent a group of formula (III), or a group —OR_{9″} where R_{9″} represents a protective group for a hydroxyl function,
R₅ represents a hydrogen atom, a group of formula (III), or a group —OR_{9′″} where R_{9′″} represents a protective group for a hydroxyl function,
R₆ represents a group of formula (III), or a group —OR_{9′″} where R_{9′″} represents a protective group for a hydroxyl function, and
R represents a furan nucleus, which may optionally be substituted,
at least one substituent among R₁, R₂, R₃, R₄, R₅, R₆, R₈, R″₁, R″₂ and R″₃ representing a group of formula (III), or
a salt thereof.

2. The compound comprising one or more epoxide function(s) as claimed in claim 1, wherein at least two substituents among R₁, R₂, R₃, R₄, R₅, R₆, R₈, R″₁, R″₂ and R″₃ represent a group of formula (III).

3. A synthesis method for synthesizing a compound having one or more epoxide function(s) as claimed in claim 1, further comprising a step of epoxidation of a compound of general formula (V): 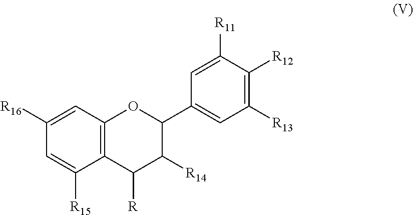
wherein:
R₁₁, R₁₂, R₁₃ and R₁₅, which is identical or different, each represent a hydrogen atom or a hydroxyl group, optionally protected with a protective group,
R₁₄ represents a hydrogen atom or a group —OR₁₇, wherein R₁₇ represents a hydrogen atom, a protective group for a hydroxyl function or a group of general formula (II′): 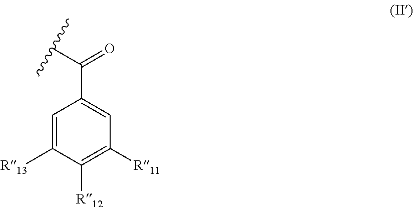
wherein R″₁₁, R″₁₂ and R″₁₃, which is identical or different, each represent a hydroxyl group, optionally protected with a protective group,
R₁₆ represents a hydroxyl group, optionally protected with a protective group, and
R represents a furan nucleus, optionally substitutable,
at least one substituent among R₁₁, R₁₂, R₁₃, R₁₄, R₁₅, R₁₆, R″₁₁, R″₁₂ and R″₁₃ representing a hydroxyl group, or
a salt thereof.

4. The synthesis method as claimed in claim 3, wherein the epoxidation of the compound of general formula (V) is carried out by placing said compound of general formula (V) in the presence of an epihalohydrin.

5. A synthesis method as claimed in claim 3, wherein the compound of general formula (V) is obtained by a reaction of depolymerization of condensed tannins in the presence of an acid by means of a nucleophile of general formula (VII): 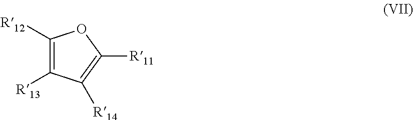
wherein R′₁₁, R′₁₂, R′₁₃ and R′₁₄, which is identical or different, each represent a hydrogen atom or a substituent not comprising a mesomeric-effect electron-withdrawing group conjugated to the furan nucleus, at least one substituent among R′₁₁, R′₁₂, R′₁₃ and R′₁₄ representing a hydrogen atom.

6. The synthesis method as claimed in claim 5, wherein the epoxidation step is carried out on the reaction crude obtained by the reaction of condensed-tannin depolymerization in the presence of an acid by means of the nucleophile of general formula (VII).

7. A method for producing an epoxy resin using a compound having one or more epoxide function(s) as claimed in claim 1.

8. A method for producing an epoxy resin using a compound having one or more epoxide function(s) as claimed in claim 2, and constitutes a precursor synthon for said epoxy resin.

9. A method for producing an epoxy resin using a compound having one or more epoxide function(s) as claimed in claim 2, and is subjected, in order to form a compound constituting a precursor synthon for said epoxy resin, to a step of opening at least one of its epoxide rings by reaction with a nucleophilic reagent having a function capable of reacting with said epoxide ring so as to cause opening thereof, with an exclusion of an amine function, and having no other group that is reactive with respect to the epoxide functions, said step being carried out to leave intact at least one epoxide ring of said compound having one or more epoxide function(s).

10. The method as claimed in claim 7, wherein the compound having one or more epoxide function(s) is subjected to a step of opening at least one of its epoxide rings with introduction of an amine function, so as to form a curing agent for the production of said epoxy resin.

11. A method for producing an epoxy resin, by mixing at least one epoxide prepolymer and a curing agent in accordance with claim 2, wherein at least one of:
said epoxide prepolymer is a compound comprising epoxide functions, or is obtained from a compound comprising epoxide functions by a step of opening at least one of its epoxide rings by reaction with a nucleophilic reagent having a function capable of reacting with said epoxide ring so as to cause opening thereof, with an exclusion of an amine function, and having no other group that is reactive with respect to the epoxide functions, said step being carried out so as to leave intact at least one epoxide ring of said compound having one or more epoxide function(s); and
said curing agent is obtained from a compound having one or more epoxide function(s) by a step of opening at least one epoxide ring of said compound having one or more epoxide function(s) with introduction of an amine function.

12. The method for producing the epoxy resin as claimed in claim 11, wherein the step of opening at least one epoxide ring of the compound having one or more epoxide function(s) with an introduction of an amine function is carried out by placing said compound having one or more epoxide function(s) in the presence of a compound, called functionalization compound, comprising a first function capable of reacting with the epoxide ring so as to cause opening thereof, and a second function being a primary amine function, or in a presence of ammonia.

13. The method for producing an epoxy resin as claimed in claim 12, wherein the functionalization compound is cysteamine.

14. The method for producing an epoxy resin as claimed in claim 11, wherein the step of opening at least one epoxide ring of said compound having one or more epoxide function(s) with an introduction of an amine function is preceded by, or carried out simultaneously with, a step of opening at least one of the epoxide rings of said compound having one or more epoxide function(s) by reaction with a nucleophilic reagent having a function capable of reacting with said epoxide ring so as to cause opening thereof, with the exclusion of an amine function, and having no other group that is reactive with respect to the epoxide functions, said step being carried out so as to leave intact at least one epoxide ring of said compound having one or more epoxide function(s) for the step of opening said epoxide ring with the introduction of an amine function.

15. An epoxy resin obtained by the method as claimed in claim 11.

16. The epoxy resin as claimed in claim 15 to obtain materials for insulating electrical and/or electronic components.

17. The epoxy resin as claimed in claim 15 to manufacture materials to be in contact with food.

18. A compound having one or more amine function(s) obtained by a step of opening at least one epoxide ring of a compound having one or more epoxide function(s) as claimed in claim 1 with an introduction of an amine function, or a salt thereof.

19. The compound having one or more amine function(s) as claimed in claim 18, corresponding to general formula (VIII): 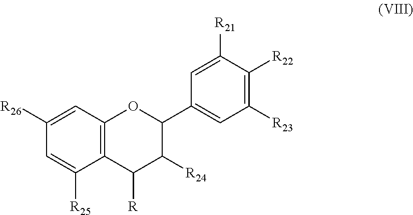
wherein:
R₂₁, R₂₂ and R₂₃, which is identical or different, each represent a hydrogen atom, a group of formula (IX) below, or a group —OR₂₉ where R₂₉ represents a protective group for a hydroxyl function, and
(R₂₁ and R₂₂) or (R₂₂ and R₂₃) together represent a group of general formula (X): 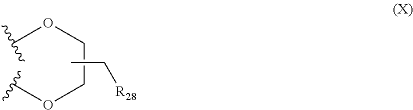
wherein R₂₈ represents a hydroxyl group, a group of formula (IX) below, or a group —OR_{29′} where R_{29′} represents a protective group for a hydroxyl function,
R₂₄ represents a hydrogen atom, a hydroxyl group, optionally protected with a protective group, a group of formula (IX) below or a group —OR₂₇, wherein R₂₇ represents a group of general formula (XI): 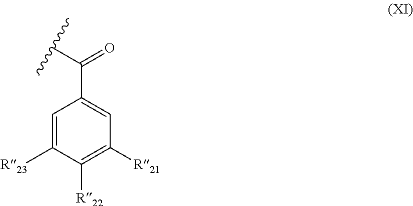
wherein R″₂₁, R″₂₂ and R″₂₃, which is identical or different, each represent a group of formula (IX) below, or a group —OR_{29″} where R_{29″} represents a protective group for a hydroxyl function,
R₂₅ represents a hydrogen atom, a group of formula (IX) below, or a group —OR_{29′″} where R_{29′″} represents a protective group for a hydroxyl function,
R₂₆ represents a group of formula (IX) below, or a group —OR_{29′″}, where R_{29′″} represents a protective group for a hydroxyl function, and
R represents a furan nucleus, optionally substitutable,
at least one substituent among R₂₁, R₂₂, R₂₃, R₂₄, R₂₅, R₂₆, R₂₈, R″₂₁, R″₂₂ or R″₂₃ representing a group of formula (IX) below,
said group of formula (IX) being chosen from the groups: 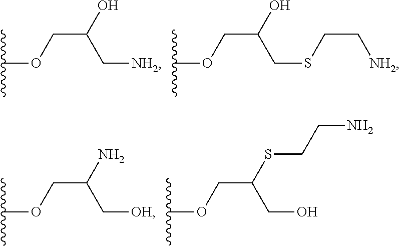
or a salt thereof.
